(19) United States
(12) Patent Application Publication
(10) Pub. No.: US 2014/0199405 A1
(43) Pub. Date: Jul. 17, 2014

(54) METHOD FOR TREATING CANCER BASED ON MUTATION STATUS OF K-RAS

(71) Applicant: Abraxis BioScience, LLC, Los Angeles, CA (US)

(72) Inventors: Daniel PIERCE, Summit, NJ (US); Carrie BRACHMANN, Summit, NJ (US)

(21) Appl. No.: 13/794,712

(22) Filed: Mar. 11, 2013

Related U.S. Application Data
(60) Provisional application No. 61/848,793, filed on Jan. 11, 2013.
(60) Provisional application No. 61/752,417, filed on Jan. 14, 2013.

(51) Int. Cl. A61K 9/51; A61K 31/7068; A61K 31/337

(52) U.S. Cl. CPC A61K 9/5169; A61K 31/337; A61K 31/7068
USPC 424/491; 514/449; 514/49; 424/130.1

(57) ABSTRACT

The present invention provides methods and compositions for treating cancer by administering a) a composition comprising nanoparticles that comprise paclitaxel and an albumin and/or b) a therapeutic agent (e.g., gemcitabine) based upon K-ras mutation status.

### RELATED APPLICATIONS

This application claims priority from U.S. Provisional Patent Application No. 61/848,793, filed Jan. 11, 2013, and U.S. Provisional Patent Application No. 61/752,417, filed Jan. 14, 2013 the contents of which are incorporated herein by reference in its entirety.

### TECHNICAL FIELD

The present invention relates to methods and compositions for determining responsiveness and/or likelihood of successful treatment comprising administering compositions comprising nanoparticles that comprise taxane (e.g., paclitaxel) and an albumin.

### BACKGROUND

Cancer is the leading cause of death in the United States. Pancreatic cancer has one of the highest mortality rates among all cancers and is expected to cause an estimated 37,390 deaths in the United States in 2012. See Cancer Facts and Figures, American Cancer Society (2012). For all stages of pancreatic cancer combined, the 1- and 5-year relative survival rates are 26% and 6%, respectively. This high mortality rate from pancreatic cancer is, at least in part, due to the high incidence of metastatic disease at the time of diagnosis. As a result, treatment options for pancreatic cancer are very limited.

The standard first-line treatment for treating pancreatic cancer is gemcitabine (e.g., GEMZAR®), which was approved by the Food and Drug Administration (“FDA”) in 1996. In a clinical study with 126 patients with locally advanced pancreatic cancer (63 treated with gemcitabine), gemcitabine was shown to be superior to 5-fluororuracil (5-FU) in terms of median overall survival (5.7 months for gemcitabine versus 4.2 months for 5-FU), median time to disease progression (2.1 months for gemcitabine versus 0.9 months for 5-FU), and clinical benefit responses. However, although gemcitabine has become a standard palliative therapy for treating pancreatic cancer since its approval in 1996, there has been little improvement in pancreatic cancer treatment.

The gemcitabine/erlotinib combination improved the median overall survival (6.4 months versus 6.0 months) and median progression free survival (3.8 months versus 3.5 months) over gemcitabine monotherapy. See Moore et al., J. Clin. Oncol. 25:1960-1966 (2007). Based on this very modest improvement in overall survival and progression free survival (0.4 and 0.3 months, respectively), the FDA approved the gemcitabine/erlotinib combination in 2005. Despite its approval, the gemcitabine/erlotinib combination has not been widely used as a standard of care for treating pancreatic cancer because of side effects associated with the gemcitabine/erlotinib combination and the minimal improvement on survival over gemcitabine monotherapy. See Nieto et al., The Oncologist, 13:562-576 (2008).

Albumin-based nanoparticle compositions have been developed as a drug delivery system for delivering substantially water insoluble drugs such as a taxanes. See, for example, U.S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as pancreatic cancer and melanoma.

Albumin-based paclitaxel nanoparticle compositions (e.g., Abraxane®) in combination with gemcitabine was found to be well tolerated in advanced pancreatic cancer in a Phase I/II study and showed evidence of antitumor activity. See, for example, US Patent App.; No. 2006/0263434; Maitra et al., Mol. Cancer Ther. 8(12 Suppl): C246 (2009); Loehr et al., J. of Clinical Oncology 27 (15S) (May 20 Supplement):200, Abstract No. 4526 (2009); Von Hoff et al., J. of Clinical Oncology 27(15S)(May 20 Supplement), Abstract No. 4525 (2009); and Kim et al., Proc. Amer. Assoc. Cancer Res., 46, Abstract No. 1440 (2005).

Oncogenic K-ras mutations have been identified in a variety of cancers including pancreatic cancer. Laghi et al., Oncogene., 2002, 21:4301-4306; Jarell et al., Biologics., 2007, 1(4):407-14; Fernandez-Medarde et al., Genes Cancer., 2011, 2(3):344-58. K-ras mutations have been studied as a marker for treating pancreatic cancer with gemcitabine monotherapy or combination therapy, however a beneficial response rate to gemcitabine therapy according to K-ras mutation status was not observed. Kim et al., Mol. Cancer Ther., 2011, 10(10):1993-1999.

The disclosures of all publications, patents, patent applications and published patent applications referred to herein are hereby incorporated herein by reference in their entirety.

### BRIEF SUMMARY OF THE INVENTION

The present application provides a method of treating cancer in an individual (such as a human individual) comprising administering (such as intravenously administering) to the individual an effective amount of a composition comprising nanoparticles comprising a taxane (such as paclitaxel) and an albumin, wherein the individual has a K-ras mutation. In some embodiments, there is provided a method of treating cancer in an individual (such as human individual) comprising administering (such as intravenously administering) to the individual an effective amount of a composition comprising nanoparticles comprising a taxane (such as paclitaxel) and an albumin, wherein the K-ras mutation status is used as a basis for selecting the individual for treatment. In some embodiments, the individual is selected for treatment if the individual has a K-ras mutation. In some embodiments, the K-ras mutation is detected in a K-ras amino acid sequence. In some embodiments, the K-ras mutation is detected in a nucleic acid encoding K-ras protein.

In some embodiments according to any of the embodiments above, the method further comprises administering to the individual an effective amount of a therapeutic agent (such as gemcitabine). In some embodiments, the composition comprising nanoparticles comprising a taxane and albumin and the therapeutic agent are administered sequentially. In some embodiments, the composition comprising nanoparticles comprising a taxane and albumin and the therapeutic agent are administered simultaneously.

In some embodiments according to any of the embodiments above, the nanoparticles in the composition comprises the taxane coated with the albumin. In some embodiments, the nanoparticles in the composition have an average diameter of less than about 200 nm. In some embodiments, the composition is nab-paclitaxel (Abraxane®). In some embodiments according to any of the embodiments above, the method further comprises determining the K-ras mutation status prior to administering to the individual an effective amount of the composition comprising nanoparticles comprising a taxane and an albumin. In some embodiments, the method further comprises comparing the K-ras mutation status with a control. In some embodiments, the individual is selected for treatment if the individual has a K-ras mutation. In further embodiments, the K-ras mutation is at one or more of G12, G13 S17, P34 or Q61 of the K-ras amino acid sequence (position corresponding to the K-ras sequence of SEQ ID NO:2). In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H.

In another aspect, there is provided a kit comprising 1) a composition comprising nanoparticles comprising a taxane (such as paclitaxel) and an albumin, and 2) an agent for determining the K-ras mutation status.

Also provided are compositions (such as pharmaceutical compositions), medicine, kits, and unit dosages useful for methods described herein.

These and other aspects and advantages of the present invention will become apparent from the subsequent detailed description and the appended claims. It is to be understood that one, some, or all of the properties of the various embodiments described herein may be combined to form other embodiments of the present invention.

### DETAILED DESCRIPTION OF THE INVENTION

The present invention provides methods of treatment with an albumin-based taxane nanoparticle composition based on the K-ras mutation status. In one aspect, there is provided a method of treating cancer in an individual having a K-ras mutation by administering to the individual an effective amount of a composition comprising nanoparticles comprising a taxane (such as paclitaxel) and albumin, and in some embodiments, further administering an effective amount of a therapeutic agent (such as gemcitabine).

In another aspect, there is provided a method of treating cancer in an individual by administering to the individual an effective amount of a composition comprising nanoparticles comprising a taxane (such as paclitaxel) and albumin, and in some embodiments, further administering an effective amount of a therapeutic agent (such as gemcitabine), wherein the individual is selected for treatment based on the K-ras mutation status.

In another aspect, there is provided a method of selecting (including identifying) an individual for treating with a composition comprising nanoparticles comprising a taxane (such as paclitaxel) and albumin, and in some embodiments, an effective amount of a therapeutic agent (such as gemcitabine), wherein the method comprises determining the K-ras mutation status.

The K-ras mutation status can also be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits.

Also provided are compositions (such as pharmaceutical compositions), medicine, kits, and unit dosages useful for the methods described herein.

### DEFINITIONS

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “individual” refers to a mammal and includes, but is not limited to, human, bovine, horse, feline, canine, rodent, or primate. In some embodiments, the individual is a human.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer. An individual having one or more of these risk factors has a higher probability of developing cancer than an individual without these risk factor(s).

“Adjuvant setting” refers to a clinical setting in which an individual has had a history of cancer, and generally (but not necessarily) been responsive to therapy, which includes, but is not limited to, surgery (e.g., surgery resection), radiotherapy, and chemotherapy. However, because of their history of cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

As used herein, by “combination therapy” is meant that a first agent be administered in conjunction with another agent. “In conjunction with” refers to administration of one treatment modality in addition to another treatment modality, such as administration of a nanoparticle composition described herein in addition to administration of the other agent to the same individual. As such, “in conjunction with” refers to administration of one treatment modality before, during, or after delivery of the other treatment modality to the individual. Such combinations are considered to be part of a single treatment regimen.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

An “adverse event” or “AE” as used herein refers to any untoward medical occurrence in an individual receiving a marketed pharmaceutical product or in an individual who is participating on a clinical trial who is receiving an investigational or non-investigational pharmaceutical agent. The AE does not necessarily have a causal relationship with the individual's treatment. Therefore, an AE can be any unfavorable and unintended sign, symptom, or disease temporally associated with the use of a medicinal product, whether or not considered to be related to the medicinal product. An AE includes, but is not limited to: an exacerbation of a pre-existing illness; an increase in frequency or intensity of a pre-existing episodic event or condition; a condition detected or diagnosed after study drug administration even though it may have been present prior to the start of the study; and continuously persistent disease or symptoms that were present at baseline and worsen following the start of the study. An AE generally does not include: medical or surgical procedures (e.g., surgery, endoscopy, tooth extraction, or transfusion); however, the condition that leads to the procedure is an adverse event; pre-existing diseases, conditions, or laboratory abnormalities present or detected at the start of the study that do not worsen; hospitalizations or procedures that are done for elective purposes not related to an untoward medical occurrence (e.g., hospitalizations for cosmetic or elective surgery or social/convenience admissions); the disease being studied or signs/symptoms associated with the disease unless more severe than expected for the individual's condition; and overdose of study drug without any clinical signs or symptoms.

A “serious adverse event” or (SAE) as used herein refers to any untoward medical occurrence at any dose including, but not limited to, that: a) is fatal; b) is life-threatening (defined as an immediate risk of death from the event as it occurred); c) results in persistent or significant disability or incapacity; d) requires in-patient hospitalization or prolongs an existing hospitalization (exception: Hospitalization for elective treatment of a pre-existing condition that did not worsen during the study is not considered an adverse event. Complications that occur during hospitalization are AEs and if a complication prolongs hospitalization, then the event is serious); e) is a congenital anomaly/birth defect in the offspring of an individual who received medication; or f) conditions not included in the above definitions that may jeopardize the individual or may require intervention to prevent one of the outcomes listed above unless clearly related to the individual's underlying disease. “Lack of efficacy” (progressive disease) is not considered an AE or SAE. The signs and symptoms or clinical sequelae resulting from lack of efficacy should be reported if they fulfill the AE or SAE definitions.

The following definitions may be used to evaluate response based on target lesions: “complete response” or “CR” refers to disappearance of all target lesions; “partial response” or “PR” refers to at least a 30% decrease in the sum of the longest diameters (SLD) of target lesions, taking as reference the baseline SLD; “stable disease” or “SD” refers to neither sufficient shrinkage of target lesions to qualify for PR, nor sufficient increase to qualify for PD, taking as reference the nadir SLD since the treatment started; and “progressive disease” or “PD” refers to at least a 20% increase in the SLD of target lesions, taking as reference the nadir SLD recorded since the treatment started, or, the presence of one or more new lesions.

The following definitions of response assessments may be used to evaluate a non-target lesion: “complete response” or “CR” refers to disappearance of all non-target lesions; “stable disease” or “SD” refers to the persistence of one or more non-target lesions not qualifying for CR or PD; and “progressive disease” or “PD” refers to the “unequivocal progression” of existing non-target lesion(s) or appearance of one or more new lesion(s) is considered progressive disease (if PD for the subject is to be assessed for a time point based solely on the progression of non-target lesion(s), then additional criteria are required to be fulfilled.

“Progression free survival” (PFS) indicates the length of time during and after treatment that the cancer does not grow. Progression-free survival includes the amount of time individuals have experienced a complete response or a partial response, as well as the amount of time individuals have experienced stable disease.

A “complete response” (CR) to a therapy defines individuals with evaluable but non-measurable disease, whose tumor and all evidence of disease had disappeared.

A “partial response” (PR) to a therapy defines individuals with anything less than complete response were simply categorized as demonstrating partial response.

“Stable disease” (SD) indicates that the individual is stable.

“Correlate” or “correlating” is meant comparing, in any way, the performance and/or results of a first analysis or protocol with the performance and/or results of a second analysis or protocol. For example one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

“Predicting” or “prediction” is used herein to refer to the likelihood that an individual is likely to respond either favorably or unfavorably to a treatment regimen.

As used herein, “at the time of starting treatment” or “baseline” refers to the time period at or prior to the first exposure to the treatment.

A method of “aiding assessment” as used herein refers to methods that assist in making a clinical determination and may or may not be conclusive with respect to the assessment.

“Likely to respond” or “responsiveness” as used herein refers to any kind of improvement or positive response either clinical or non-clinical selected from, but not limited to, measurable reduction in tumor size or evidence of disease or disease progression, complete response, partial response, stable disease, increase or elongation of progression free survival, or increase or elongation of overall survival.

As used herein, “sample” refers to a composition which contains a molecule which is to be characterized and/or identified, for example, based on physical, biochemical, chemical, physiological, and/or genetic characteristics.

“Cells,” as used herein, is understood to refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein.

The K-ras mutation status determined “before or upon initiation of treatment” is the K-ras mutation status determined in an individual before the individual receives the first administration of a treatment modality described herein.

An individual who “may be suitable”, which includes an individual who is “suitable” for treatment(s) described herein, is an individual who is more likely than not to benefit from administration of said treatments. Conversely, an individual who “may not be suitable” or “may be unsuitable”, which includes an individual who is “unsuitable” for treatment(s) described herein, is an individual who is more likely than not to fail to benefit from administration of said treatments.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise.

As is apparent to one skilled in the art, an individual assessed, selected for, and/or receiving treatment is an individual in need of such activities.

### Methods of Treating

The present invention in one embodiment provides a method of treating cancer (such as pancreatic cancer) in an individual by administering to the individual an effective amount of a composition comprising nanoparticles comprising a taxane (such as paclitaxel) and albumin, wherein the individual is selected for treatment based on the K-ras mutation status (such as K-ras G12 mutation status, i.e., a mutation at the G12 position corresponding to SEQ ID NO:2). In some embodiments, there is provided a method of treating cancer in an individual by administering to the individual i) an effective amount of a composition comprising nanoparticles comprising a taxane (such as paclitaxel) and albumin, and ii) an effective amount of a therapeutic agent (such as gemcitabine), wherein the individual is selected for treatment based on the K-ras mutation status (such as K-ras G12 mutation status). In some embodiments, there is provided a method of treating cancer in an individual comprising administering to the individual (i) an effective amount of a composition comprising nanoparticles comprising paclitaxel coated with albumin (including nanoparticles having an average diameter of no greater than about 200 nm); and (ii) an effective amount of a therapeutic agent (such as gemcitabine), wherein the individual is selected for treatment based on the K-ras mutation status (such as K-ras G12 mutation status). In some embodiments, there is provided a method of treating cancer (such as pancreatic cancer) in a human individual comprising administering to the individual (i) an effective amount of nab-paclitaxel (for example about 5 mg/ml nab-paclitaxel); and (ii) an effective amount of gemcitabine, wherein the individual is selected for treatment based on the K-ras mutation status (such as K-ras G12 mutation status). In some embodiments, the K-ras mutation status is determined in a K-ras amino acid sequence. In some embodiments, the K-ras mutation is determined in a nucleic acid encoding K-ras protein. In some embodiments, the K-ras mutation status is determined (e.g., K-ras G12 mutation status) by comparing to a control (such as any of the controls described herein). In some embodiments, the individual having wild-type K-ras is not selected for treatment. In some embodiments, the individual having a K-ras mutation is selected for treatment. In further embodiments, the K-ras mutation is at one or more of G12, G13 S17, P34 or Q61 of the K-ras amino acid sequence. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H.

As used herein, “based upon” includes assessing, determining, or measuring the individual's characteristics as described herein (and preferably selecting an individual suitable for receiving treatment). When a K-ras mutation status is “used as a basis” for selection, assessing, measuring, or determining method of treatment as described herein, the K-ras mutation status is determined before and/or during treatment, and the status obtained is used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

In some embodiments, there is provided a method of treating cancer (such as pancreatic cancer) in an individual by administering to the individual an effective amount of a composition comprising nanoparticles comprising a taxane (such as paclitaxel) and albumin, wherein the individual is selected for treatment based on the individual having a K-ras mutation (such as K-ras G12 mutation). In some embodiments, there is provided a method of treating cancer (such as pancreatic cancer) in an individual by administering to the individual i) an effective amount of a composition comprising nanoparticles comprising a taxane (such as paclitaxel) and albumin, and ii) an effective amount of a therapeutic agent (such as gemcitabine), wherein the individual is selected for treatment based on the individual having a K-ras mutation (such as K-ras G12 mutation). In some embodiments, there is provided a method of treating cancer (such as pancreatic cancer) in an individual comprising administering to the individual (i) an effective amount of a composition comprising nanoparticles comprising paclitaxel coated with albumin (including nanoparticles having an average diameter of no greater than about 200 nm); and (ii) an effective amount of a therapeutic agent (such as gemcitabine), wherein the individual is selected for treatment based on the individual having a K-ras mutation (such as K-ras G12 mutation). In some embodiments, there is provided a method of treating cancer (such as pancreatic cancer) in a human individual comprising administering to the individual (i) an effective amount of nab-paclitaxel (for example about 5 mg/ml nab-paclitaxel); and (ii) an effective amount of gemcitabine, wherein the individual is selected for treatment based on the individual having a K-ras mutation (such as K-ras G12 mutation). In some embodiments, the K-ras mutation status is determined in a K-ras amino acid sequence. In some embodiments, the K-ras mutation is determined in a nucleic acid encoding K-ras protein. In some embodiments, the K-ras mutation status is determined (e.g., K-ras G12 mutation status) by comparing to a control (such as any of the controls described herein). In further embodiments, the K-ras mutation is at one or more of G12, G13 S17, P34 or Q61 of the K-ras amino acid sequence. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In yet other further embodiments, the K-ras mutation is at one or more of G12, G13, Q61, or F156 of the K-ras amino acid sequence. In some embodiments, the K-ras mutation is one or more selected from the group consisting of G12C, G12R, G12S, G12D, G12V, G13D, Q61K, Q61R, Q61L, Q61H, and F156L. In yet other further embodiments, the K-ras mutation is at one or more of G12, G13, or Q61 of the K-ras amino acid sequence. In some embodiments, the K-ras mutation is one or more selected from the group consisting of G12C, G12R, G12A, G12D, G12V, G13D, and Q61L. In yet other further embodiments, the K-ras mutation is at one or more of G12 or G13 of the K-ras amino acid sequence. In some embodiments, the K-ras mutation is one or more selected from the group consisting of G12C, G12R, G12A, G12D, G12V, and G13D.

In some embodiments, there is provided a method of treating cancer (such as pancreatic cancer) in an individual, comprising: (a) assessing the K-ras mutation status in the individual; and (b) administering to the individual an effective amount of a composition comprising nanoparticles comprising a taxane (such as paclitaxel) and albumin, wherein the individual is selected for treatment based on the individual having a K-ras mutation (such as K-ras G12 mutation). In some embodiments, there is provided a method of treating cancer (such as pancreatic cancer) in an individual, comprising: (a) assessing the K-ras mutation status in the individual; and (b) administering to the individual i) an effective amount of a composition comprising nanoparticles comprising a taxane (such as paclitaxel) and albumin, and ii) an effective amount of a therapeutic agent (such as gemcitabine), wherein the individual is selected for treatment based on the individual having a K-ras mutation (such as K-ras G12 mutation). In some embodiments, there is provided a method of treating cancer (such as pancreatic cancer) in an individual comprising (a) assessing the K-ras mutation status of the individual; and (b) administering to the individual (i) an effective amount of a composition comprising nanoparticles comprising paclitaxel coated with albumin (including nanoparticles having an average diameter of no greater than about 200 nm); and (ii) an effective amount of a therapeutic agent (such as gemcitabine), wherein the individual is selected for treatment based on the individual having a K-ras mutation (such as K-ras G12 mutation). In some embodiments, there is provided a method of treating cancer (such as pancreatic cancer) in a human individual comprising: (a) assessing the K-ras mutation status of the individual; and (b) administering to the individual (i) an effective amount of nab-paclitaxel (for example about 5 mg/ml nab-paclitaxel); and (ii) an effective amount of gemcitabine, wherein the individual is selected for treatment based on the individual having a K-ras mutation (such as K-ras G12 mutation). In some embodiments, the K-ras mutation status is determined in a K-ras amino acid sequence. In some embodiments, the K-ras mutation is determined in a nucleic acid encoding K-ras protein. In some embodiments, the K-ras mutation status is determined (e.g., K-ras G12 mutation status) by comparing to a control (such as any of the controls described herein). In further embodiments, the K-ras mutation is at one or more of G12, G13 S17, P34 or Q61 of the K-ras amino acid sequence. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In yet other further embodiments, the K-ras mutation is at one or more of G12, G13, Q61, or F156 of the K-ras amino acid sequence. In some embodiments, the K-ras mutation is one or more selected from the group consisting of G12C, G12R, G12S, G12D, G12V, G13D, Q61K, Q61R, Q61L, Q61H, and F156L. In yet other further embodiments, the K-ras mutation is at one or more of G12, G13, or Q61 of the K-ras amino acid sequence. In some embodiments, the K-ras mutation is one or more selected from the group consisting of G12C, G12R, G12A, G12D, G12V, G13D, and Q61L. In yet other further embodiments, the K-ras mutation is at one or more of G12 or G13 of the K-ras amino acid sequence. In some embodiments, the K-ras mutation is one or more selected from the group consisting of G12C, G12R, G12A, G12D, G12V, and G13D.

In some embodiments, there is provided a method of treating cancer (such as pancreatic cancer) in an individual, comprising: (a) assessing the K-ras mutation status in the individual; (b) selecting (e.g., identifying) the individual for treatment based on the individual having a K-ras mutation (such as K-ras G12 mutation); and (c) administering to the individual an effective amount of a composition comprising nanoparticles comprising a taxane (such as paclitaxel) and albumin. In some embodiments, there is provided a method of treating cancer (such as pancreatic cancer) in an individual, comprising: (a) assessing the K-ras mutation status in the individual; (b) selecting (e.g., identifying) the individual for treatment based on the individual having a K-ras mutation (such as K-ras G12 mutation); and (c) administering to the individual i) an effective amount of a composition comprising nanoparticles comprising a taxane (such as paclitaxel) and albumin, and ii) an effective amount of a therapeutic agent (such as gemcitabine). In some embodiments, there is provided a method of treating cancer (such as pancreatic cancer) in an individual comprising (a) assessing the K-ras mutation status of the individual; (b) selecting (e.g., identifying) the individual for treatment based on the individual having a K-ras mutation (such as K-ras G12 mutation); and (c) administering to the individual (i) an effective amount of a composition comprising nanoparticles comprising paclitaxel coated with albumin (including nanoparticles having an average diameter of no greater than about 200 nm); and (ii) an effective amount of a therapeutic agent (such as gemcitabine). In some embodiments, there is provided a method of treating cancer (such as pancreatic cancer) in a human individual comprising: (a) assessing the K-ras mutation status of the individual; (b) selecting (e.g., identifying) the individual for treatment based on the individual having a K-ras mutation (such as K-ras G12 mutation); and (c) administering to the individual (i) an effective amount of nab-paclitaxel (for example about 5 mg/ml nab-paclitaxel); and (ii) an effective amount of gemcitabine. In some embodiments, the K-ras mutation status is determined in a K-ras amino acid sequence. In some embodiments, the K-ras mutation is determined in a nucleic acid encoding K-ras protein. In some embodiments, the K-ras mutation status is determined (e.g., K-ras G12 mutation status) by comparing to a control (such as any of the controls described herein). In further embodiments, the K-ras mutation is at one or more of G12, G13 S17, P34 or Q61 of the K-ras amino acid sequence. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In yet other further embodiments, the K-ras mutation is at one or more of G12, G13, Q61, or F156 of the K-ras amino acid sequence. In some embodiments, the K-ras mutation is one or more selected from the group consisting of G12C, G12R, G12S, G12D, G12V, G13D, Q61K, Q61R, Q61L, Q61H, and F156L. In yet other further embodiments, the K-ras mutation is at one or more of G12, G13, or Q61 of the K-ras amino acid sequence. In some embodiments, the K-ras mutation is one or more selected from the group consisting of G12C, G12R, G12A, G12D, G12V, G13D, and Q61L. In yet other further embodiments, the K-ras mutation is at one or more of G12 or G13 of the K-ras amino acid sequence. In some embodiments, the K-ras mutation is one or more selected from the group consisting of G12C, G12R, G12A, G12D, G12V, and G13D.

The present invention in one embodiment provides a method of treating cancer (such as pancreatic cancer) in an individual by administering to the individual an effective amount of a composition comprising nanoparticles comprising a taxane (such as paclitaxel) and albumin, wherein the individual has a K-ras mutation (such as K-ras G12 mutation). In some embodiments, there is provided a method of treating cancer (such as pancreatic cancer) in an individual by administering to the individual i) an effective amount of a composition comprising nanoparticles comprising a taxane (such as paclitaxel) and albumin, and ii) an effective amount of a therapeutic agent (such as gemcitabine), wherein the individual has a K-ras mutation (such as K-ras G12 mutation). In some embodiments, there is provided a method of treating cancer (such as pancreatic cancer) in an individual comprising administering to the individual (i) an effective amount of a composition comprising nanoparticles comprising paclitaxel coated with albumin (including nanoparticles having an average diameter of no greater than about 200 nm); and (ii) an effective amount of a therapeutic agent (such as gemcitabine), wherein the individual has a K-ras mutation (such as K-ras G12 mutation). In some embodiments, there is provided a method of treating cancer (such as pancreatic cancer) in a human individual comprising administering to the individual (i) an effective amount of nab-paclitaxel (for example about 5 mg/ml nab-paclitaxel); and (ii) an effective amount of gemcitabine, wherein the individual has a K-ras mutation (such as K-ras G12 mutation). In some embodiments, the K-ras mutation status is determined in a K-ras amino acid sequence. In some embodiments, the K-ras mutation is determined in a nucleic acid encoding K-ras protein. In some embodiments, the K-ras mutation status is determined (e.g., K-ras G12 mutation status) by comparing to a control (such as any of the controls described herein). In some embodiments, the individual having a K-ras mutation is selected for treatment. In further embodiments, the K-ras mutation is at one or more of G12, G13 S17, P34 or Q61 of the K-ras amino acid sequence. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In yet other further embodiments, the K-ras mutation is at one or more of G12, G13, Q61, or F156 of the K-ras amino acid sequence. In some embodiments, the K-ras mutation is one or more selected from the group consisting of G12C, G12R, G12S, G12D, G12V, G13D, Q61K, Q61R, Q61L, Q61H, and F156L. In yet other further embodiments, the K-ras mutation is at one or more of G12, G13, or Q61 of the K-ras amino acid sequence. In some embodiments, the K-ras mutation is one or more selected from the group consisting of G12C, G12R, G12A, G12D, G12V, G13D, and Q61L. In yet other further embodiments, the K-ras mutation is at one or more of G12 or G13 of the K-ras amino acid sequence. In some embodiments, the K-ras mutation is one or more selected from the group consisting of G12C, G12R, G12A, G12D, G12V, and G13D.

In some embodiments, there is provided a method of selecting (including identifying) an individual having cancer (such as pancreatic cancer) for treating with a composition comprising nanoparticles comprising a taxane (such as paclitaxel) and albumin, wherein the method comprises determining the K-ras mutation status (such as K-ras G12 mutation status). In some embodiments, there is provided a method of selecting (including identifying) an individual having cancer (such as pancreatic cancer) for treating with i) a composition comprising nanoparticles comprising a taxane (such as paclitaxel) and albumin, and ii) an effective amount of a therapeutic agent (such as gemcitabine), wherein the method comprises determining the K-ras mutation status (such as K-ras G12 mutation status). In some embodiments, there is provided a method of selecting (including identifying) an individual having cancer (such as pancreatic cancer) for treating with (i) an effective amount of a composition comprising nanoparticles comprising paclitaxel coated with albumin (including nanoparticles having an average diameter of no greater than about 200 nm); and (ii) an effective amount of a therapeutic agent (such as gemcitabine), wherein the method comprises determining the K-ras mutation status (such as K-ras G12 mutation status). In some embodiments, there is provided a method of selecting (including identifying) an individual having cancer (such as pancreatic cancer) for treating with (i) an effective amount of nab-paclitaxel (for example about 5 mg/ml nab-paclitaxel), and (ii) an effective amount of gemcitabine, wherein the method comprises determining the K-ras mutation status (such as K-ras G12 mutation status). In some embodiments, the K-ras mutation status is determined in a K-ras amino acid sequence. In some embodiments, the K-ras mutation is determined in a nucleic acid encoding K-ras protein. In some embodiments, the K-ras mutation status is determined (e.g., K-ras G12 mutation status) by comparing to a control (such as any of the controls described herein). In some embodiments, the individual having a K-ras mutation is selected for treatment. In some embodiments, the individual having a wild-type K-ras is not selected for treatment. In further embodiments, the K-ras mutation is at one or more of G12, G13 S17, P34 or Q61 of the K-ras amino acid sequence. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In yet other further embodiments, the K-ras mutation is at one or more of G12, G13, Q61, or F156 of the K-ras amino acid sequence. In some embodiments, the K-ras mutation is one or more selected from the group consisting of G12C, G12R, G12S, G12D, G12V, G13D, Q61K, Q61R, Q61L, Q61H, and F156L. In yet other further embodiments, the K-ras mutation is at one or more of G12, G13, or Q61 of the K-ras amino acid sequence. In some embodiments, the K-ras mutation is one or more selected from the group consisting of G12C, G12R, G12A, G12D, G12V, G13D, and Q61L. In yet other further embodiments, the K-ras mutation is at one or more of G12 or G13 of the K-ras amino acid sequence. In some embodiments, the K-ras mutation is one or more selected from the group consisting of G12C, G12R, G12A, G12D, G12V, and G13D.

In some embodiments, there is provided a method of treating cancer (such as pancreatic cancer) in an individual comprising administering (for example intravenously) to the individual (i) an effective amount of nab-paclitaxel (for example about 5 mg/ml nab-paclitaxel); and optionally (ii) an effective amount of another therapeutic agent (such as gemcitabine), wherein the individual is selected for treatment based on a K-ras mutation. In some embodiments, there is provided a method of treating pancreatic cancer (such as metastatic pancreatic cancer or locally advanced unresectable pancreatic cancer) in an individual comprising: a) determining the K-ras mutation status of the individual, and b) administering (for example intravenously) to the individual (i) an effective amount of nab-paclitaxel (for example about 5 mg/ml nab-paclitaxel); and optionally (ii) an effective amount of another therapeutic agent (such as gemcitabine). In some embodiments, there is provided a method of treating pancreatic cancer (such as metastatic pancreatic cancer or locally advanced unresectable pancreatic cancer) in an individual comprising: a) selecting the individual for treatment based on a K-ras mutation in the individual; and b) administering (for example intravenously) to the individual (i) an effective amount of nab-paclitaxel (for example about 5 mg/ml nab-paclitaxel); and optionally (ii) an effective amount of another therapeutic agent (such as gemcitabine). In some embodiments, there is provided a method of treating pancreatic cancer (such as metastatic pancreatic cancer or locally advanced unresectable pancreatic cancer) in an individual comprising: a) assessing the K-ras mutation status of in the individual, b) selecting the individual for treatment based on a K-ras mutation in the individual; and c) administering (for example intravenously) to the individual (i) an effective amount of nab-paclitaxel (for example about 5 mg/ml nab-paclitaxel); and optionally (ii) an effective amount of another therapeutic agent (such as gemcitabine). In some embodiments, the K-ras mutation is at one or more of G12, G13 S17, P34 or Q61 of the K-ras amino acid sequence. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H.

In some embodiments, there is provided a method of treating cancer (such as lung cancer for example non-small cell lung cancer “NSCLC”) in an individual comprising administering (for example intravenously) to the individual (i) an effective amount of nab-paclitaxel (for example about 5 mg/ml nab-paclitaxel); and optionally (ii) an effective amount of another therapeutic agent (such as carboplatin), wherein the individual is selected for treatment based on a K-ras mutation. In some embodiments, there is provided a method of treating lung cancer (such as NSCLC) in an individual comprising: a) determining the K-ras mutation status of the individual, and administering (for example intravenously) to the individual (i) an effective amount of nab-paclitaxel (for example about 5 mg/ml nab-paclitaxel); and optionally (ii) an effective amount of another therapeutic agent (such as carboplatin). In some embodiments, there is provided a method of treating lung cancer (such as NSCLC) in an individual comprising: a) selecting the individual for treatment based on a K-ras mutation in the individual; and b) administering (for example intravenously) to the individual (i) an effective amount of nab-paclitaxel (for example about 5 mg/ml nab-paclitaxel); and optionally (ii) an effective amount of carboplatin. In some embodiments, there is provided a method of treating lung cancer (such as NSCLC) in an individual comprising: a) assessing the K-ras mutation status in the individual; b) selecting the individual for treatment based on a K-ras mutation in the individual; and c) administering (for example intravenously) to the individual (i) an effective amount of nab-paclitaxel (for example about 5 mg/ml nab-paclitaxel); and optionally (ii) an effective amount of another therapeutic agent (such as carboplatin). In some embodiments, the K-ras mutation is at one or more of G12, G13 or Q61 of the K-ras amino acid sequence. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H.

In some embodiments, there is provided a method of treating cancer (such as colorectal cancer) in an individual comprising administering (for example intravenously) to the individual (i) an effective amount of nab-paclitaxel (for example about 5 mg/ml nab-paclitaxel); and optionally (ii) an effective amount of another therapeutic agent (such as 5-FU (fluorouracil), capecitabine, oxaliplatin, or irinotecan), wherein the individual is selected for treatment based on a K-ras mutation. In some embodiments, there is provided a method of treating colorectal cancer in an individual comprising: a) determining the K-ras mutation status of the individual, and administering (for example intravenously) to the individual (i) an effective amount of nab-paclitaxel (for example about 5 mg/ml nab-paclitaxel); and optionally (ii) an effective amount of another therapeutic agent (such as 5-FU (fluorouracil), capecitabine, oxaliplatin, or irinotecan). In some embodiments, there is provided a method of treating colorectal cancer in an individual comprising: a) selecting the individual for treatment based on a K-ras mutation in the individual; and b) administering (for example intravenously) to the individual (i) an effective amount of nab-paclitaxel (for example about 5 mg/ml nab-paclitaxel); and optionally (ii) an effective amount of another therapeutic agent (such as 5-FU (fluorouracil), capecitabine, oxaliplatin, or irinotecan). In some embodiments, there is provided a method of treating colorectal cancer in an individual comprising: a) assessing the K-ras status of the individual; b) selecting the individual for treatment based on a K-ras mutation in the individual; and c) administering (for example intravenously) to the individual (i) an effective amount of nab-paclitaxel (for example about 5 mg/ml nab-paclitaxel); and optionally (ii) an effective amount of another therapeutic agent (such as 5-FU (fluorouracil), capecitabine, oxaliplatin, or irinotecan). In some embodiments, the K-ras mutation is at one or more of G12, G13, Q61, K117 or A146 of the K-ras amino acid sequence. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V.

In some embodiments, there is provided a method of treating cancer (such as melanoma) in an individual comprising administering (for example intravenously) to the individual (i) an effective amount of nab-paclitaxel (for example about 5 mg/ml nab-paclitaxel); and optionally (ii) an effective amount of another therapeutic agent (such as bevacizumab), wherein the individual is selected for treatment based on a K-ras mutation. In some embodiments, there is provided a method of treating melanoma (such as metastatic melanoma) in an individual comprising: a) determining the K-ras mutation status of the individual, and administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, there is provided a method of treating melanoma (such as metastatic melanoma) in an individual comprising: a) selecting the individual for treatment based on a K-ras mutation in the individual; and b) administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, there is provided a method of treating melanoma (such as metastatic melanoma) in an individual comprising: a) assessing the K-ras status of the individual; b) selecting the individual for treatment based on a K-ras mutation in the individual; and c) administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, the K-ras mutation is at one or more of G12, G13, Q61, or F156 of the K-ras amino acid sequence. In some embodiments, the K-ras mutation is one or more selected from the group consisting of G12C, G12R, G12S, G12D, G12V, G13D, Q61K, Q61R, Q61L, Q61H, and F156L.

In some embodiments, there is provided a method of treating cancer (such as breast cancer) in an individual comprising administering (for example intravenously) to the individual (i) an effective amount of nab-paclitaxel (for example about 5 mg/ml nab-paclitaxel); and optionally (ii) an effective amount of another therapeutic agent (such as bevacizumab), wherein the individual is selected for treatment based on a K-ras mutation. In some embodiments, there is provided a method of treating breast cancer (such as metastatic breast cancer) in an individual comprising: a) determining the K-ras mutation status of the individual, and administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, there is provided a method of treating breast cancer (such as metastatic breast cancer) in an individual comprising: a) selecting the individual for treatment based on a K-ras mutation in the individual; and b) administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, there is provided a method of treating breast cancer (such as metastatic breast cancer) in an individual comprising: a) assessing the K-ras status of the individual; b) selecting the individual for treatment based on a K-ras mutation in the individual; and c) administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, the K-ras mutation is at one or more of G12, G13, or Q61 of the K-ras amino acid sequence. In some embodiments, the K-ras mutation is one or more selected from the group consisting of G12C, G12R, G12A, G12D, G12V, G13D, and Q61L.

In some embodiments, there is provided a method of treating cancer (such as ovarian cancer) in an individual comprising administering (for example intravenously) to the individual (i) an effective amount of nab-paclitaxel (for example about 5 mg/ml nab-paclitaxel); and optionally (ii) an effective amount of another therapeutic agent (such as bevacizumab), wherein the individual is selected for treatment based on a K-ras mutation. In some embodiments, there is provided a method of treating ovarian cancer in an individual comprising: a) determining the K-ras mutation status of the individual, and administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, there is provided a method of treating ovarian cancer in an individual comprising: a) selecting the individual for treatment based on a K-ras mutation in the individual; and b) administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, there is provided a method of treating ovarian cancer in an individual comprising: a) assessing the K-ras status of the individual; b) selecting the individual for treatment based on a K-ras mutation in the individual; and c) administering (for example intravenously) to the individual (i) an effective amount of nab-paclitaxel (for example about 5 mg/ml nab-paclitaxel); and optionally (ii) an effective amount of another therapeutic agent (such as bevacizumab). In some embodiments, the K-ras mutation is at one or more of G12 or G13 of the K-ras amino acid sequence. In some embodiments, the K-ras mutation is one or more selected from the group consisting of G12C, G12R, G12A, G12D, G12V, and G13D.

The K-ras mutation status can also be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits.

In some embodiments, there is provided a method of treating cancer (such as pancreatic cancer) in an individual comprising administering to the individual a) an effective amount of a composition comprising nanoparticles comprising a taxane (such as paclitaxel) and an albumin; and optionally b) an effective amount of a therapeutic agent (such as gemcitabine), wherein treatment is based upon the K-ras mutation status at one or more of G12, G13 S17, P34 or Q61 of the K-ras amino acid sequence. In some embodiments, the individual has a K-ras mutation. In some embodiments, there is provided a method of treating pancreatic cancer (such as metastatic pancreatic cancer or locally advanced pancreatic cancer) in an individual comprising administering to the individual a) an effective amount of a composition comprising nanoparticles comprising a taxane (such as paclitaxel) and an albumin; and optionally b) an effective amount of a therapeutic agent (such as gemcitabine), wherein treatment is based upon the individual having a mutation at one or more of G12, G13 S17, P34 or Q61 of the K-ras amino acid sequence. In some embodiments, there is provided a method of treating pancreatic cancer (such as metastatic pancreatic cancer or locally advanced pancreatic cancer) in an individual comprising (a) selecting (e.g., identifying) an individual for treatment on the basis that the individual having a mutation at one or more of G12, G13 S17, P34 or Q61 of the K-ras amino acid sequence; and (b) administering to the individual a) an effective amount of a composition comprising nanoparticles comprising a taxane (such as paclitaxel) and an albumin; and optionally b) an effective amount of a therapeutic agent (such as gemcitabine). In some embodiments, there is provided a method of treating pancreatic cancer (such as metastatic pancreatic cancer or locally advanced pancreatic cancer) in an individual comprising (a) assessing the K-ras mutation status of the individual; (b) selecting (e.g., identifying) the individual for treatment on the basis that the individual having a mutation at one or more of G12, G13 S17, P34 or Q61 of the K-ras amino acid sequence; and (c) administering to the individual a) an effective amount of a composition comprising nanoparticles comprising a taxane (such as paclitaxel) and an albumin; and optionally b) an effective amount of a therapeutic agent (such as gemcitabine). In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H.

In some embodiments, there is provided a method of treating cancer (such as colorectal cancer) in an individual comprising administering to the individual a) an effective amount of a composition comprising nanoparticles comprising a taxane (such as paclitaxel) and an albumin; and optionally b) an effective amount of a therapeutic agent (such as a chemotherapeutic agent selected from the group consisting of 5-FU (fluorouracil), capecitabine, oxaliplatin, and irinotecan), wherein treatment is based upon the K-ras mutation status at one or more of G12, G13, Q61, K117 or A146 of the K-ras amino acid sequence. In some embodiments, the individual has a K-ras mutation. In some embodiments, there is provided a method of treating colorectal cancer in an individual comprising administering to the individual a) an effective amount of a composition comprising nanoparticles comprising a taxane (such as paclitaxel) and an albumin; and optionally b) an effective amount of a therapeutic agent (such as a chemotherapeutic agent selected from the group consisting of 5-FU (fluorouracil), capecitabine, oxaliplatin, and irinotecan), wherein treatment is based upon the individual having a mutation at one or more of G12, G13, Q61, K117 or A146 of the K-ras amino acid sequence. In some embodiments, there is provided a method of treating colorectal cancer in an individual comprising: (a) selecting the individual for treatment on the basis that the individual having a mutation at one or more of G12, G13, Q61, K117 or A146 of the K-ras amino acid sequencel; and (b) administering to the individual i) an effective amount of a composition comprising nanoparticles comprising a taxane (such as paclitaxel) and an albumin; and optionally ii) an effective amount of a therapeutic agent (such as a chemotherapeutic agent selected from the group consisting of 5-FU (fluorouracil), capecitabine, oxaliplatin, and irinotecan). In some embodiments, there is provided a method of treating colorectal cancer in an individual comprising: (a) assessing the K-ras mutation status of the individual; (b) selecting the individual for treatment on the basis that the individual having a mutation at one or more of G12, G13, Q61, K117 or A146 of the K-ras amino acid sequencel; and (c) administering to the individual i) an effective amount of a composition comprising nanoparticles comprising a taxane (such as paclitaxel) and an albumin; and optionally ii) an effective amount of a therapeutic agent (such as a chemotherapeutic agent selected from the group consisting of 5-FU (fluorouracil), capecitabine, oxaliplatin, and irinotecan). In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V.

In some embodiments, there is provided a method of treating cancer (such as lung cancer) in an individual comprising administering to the individual a) an effective amount of a composition comprising nanoparticles comprising a taxane (such as paclitaxel) and an albumin; and optionally b) an effective amount of a therapeutic agent (such as carboplatin), wherein treatment is based upon the K-ras mutation status at one or more of G12, G13 or Q61 of the K-ras amino acid sequence. In some embodiments, the individual has a K-ras mutation. In some embodiments, there is provided a method of treating lung cancer (such as NSCLC) in an individual comprising administering to the individual a) an effective amount of a composition comprising nanoparticles comprising a taxane (such as paclitaxel) and an albumin; and optionally b) an effective amount of a therapeutic agent (such as carboplatin), wherein treatment is based upon the individual having a mutation at one or more of G12, G13 or Q61 of the K-ras amino acid sequence. In some embodiments, there is provided a method of treating lung cancer (such as NSCLC) in an individual comprising: a) selecting the individual for treatment on the basis that the individual having a mutation at one or more of G12, G13 or Q61 of the K-ras amino acid sequence; and b) administering to the individual i) an effective amount of a composition comprising nanoparticles comprising a taxane (such as paclitaxel) and an albumin; and optionally, ii) an effective amount of a therapeutic agent (such as carboplatin). In some embodiments, there is provided a method of treating lung cancer (such as NSCLC) in an individual comprising: a) assessing the K-ras mutation status of the individual; b) selecting the individual for treatment on the basis that the individual having a mutation at one or more of G12, G13 or Q61 of the K-ras amino acid sequence; and c) administering to the individual i) an effective amount of a composition comprising nanoparticles comprising a taxane (such as paclitaxel) and an albumin; and optionally, ii) an effective amount of a therapeutic agent (such as carboplatin). In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H.

In some embodiments, there is provided a method of treating cancer (such as melanoma) in an individual comprising administering to the individual a) an effective amount of a composition comprising nanoparticles comprising a taxane (such as paclitaxel) and an albumin; and optionally b) an effective amount of a therapeutic agent (such as carboplatin), wherein treatment is based upon the K-ras mutation status at one or more of G12, G13, Q61, or F156 of the K-ras amino acid sequence. In some embodiments, the individual has a K-ras mutation. In some embodiments, there is provided a method of treating melanoma (such as metastatic melanoma) in an individual comprising administering to the individual a) an effective amount of a composition comprising nanoparticles comprising a taxane (such as paclitaxel) and an albumin; and optionally b) an effective amount of a therapeutic agent (such as bevacizumab), wherein treatment is based upon the individual having a mutation at one or more of G12, G13, Q61, or F156 of the K-ras amino acid sequence. In some embodiments, there is provided a method of treating melanoma (such as metastatic melanoma) in an individual comprising: a) selecting the individual for treatment on the basis that the individual having a mutation at one or more of G12, G13, Q61, or F156 of the K-ras amino acid sequence; and b) administering to the individual i) an effective amount of a composition comprising nanoparticles comprising a taxane (such as paclitaxel) and an albumin; and optionally, ii) an effective amount of a therapeutic agent (such as bevacizumab). In some embodiments, there is provided a method of treating melanoma (such as metastatic melanoma) in an individual comprising: a) assessing the K-ras mutation status of the individual; b) selecting the individual for treatment on the basis that the individual having a mutation at one or more of G12, G13, Q61, or F156 of the K-ras amino acid sequence; and c) administering to the individual i) an effective amount of a composition comprising nanoparticles comprising a taxane (such as paclitaxel) and an albumin; and optionally, ii) an effective amount of a therapeutic agent (such as bevacizumab). In some embodiments, the K-ras mutation is one or more selected from the group consisting of G12C, G12R, G12S, G12D, G12V, G13D, Q61K, Q61R, Q61L, Q61H, and F156L.

In some embodiments, there is provided a method of treating cancer (such as breast cancer) in an individual comprising administering to the individual a) an effective amount of a composition comprising nanoparticles comprising a taxane (such as paclitaxel) and an albumin; and optionally b) an effective amount of a therapeutic agent (such as carboplatin), wherein treatment is based upon the K-ras mutation status at one or more of G12, G13, or Q61 of the K-ras amino acid sequence. In some embodiments, the individual has a K-ras mutation. In some embodiments, there is provided a method of treating breast cancer (such as metastatic breast cancer) in an individual comprising administering to the individual a) an effective amount of a composition comprising nanoparticles comprising a taxane (such as paclitaxel) and an albumin; and optionally b) an effective amount of a therapeutic agent (such as bevacizumab), wherein treatment is based upon the individual having a mutation at one or more of G12, G13, or Q61 of the K-ras amino acid sequence. In some embodiments, there is provided a method of treating breast cancer (such as metastatic breast cancer) in an individual comprising: a) selecting the individual for treatment on the basis that the individual having a mutation at one or more of G12, G13, or Q61 of the K-ras amino acid sequence; and b) administering to the individual i) an effective amount of a composition comprising nanoparticles comprising a taxane (such as paclitaxel) and an albumin; and optionally, ii) an effective amount of a therapeutic agent (such as bevacizumab). In some embodiments, there is provided a method of treating breast cancer (such as metastatic breast cancer) in an individual comprising: a) assessing the K-ras mutation status of the individual; b) selecting the individual for treatment on the basis that the individual having a mutation at one or more of G12, G13, or Q61 of the K-ras amino acid sequence; and c) administering to the individual i) an effective amount of a composition comprising nanoparticles comprising a taxane (such as paclitaxel) and an albumin; and optionally, ii) an effective amount of a therapeutic agent (such as bevacizumab). In some embodiments, the K-ras mutation is one or more selected from the group consisting of G12C, G12R, G12A, G12D, G12V, G13D, and Q61L.

In some embodiments, there is provided a method of treating cancer (such as ovarian cancer) in an individual comprising administering to the individual a) an effective amount of a composition comprising nanoparticles comprising a taxane (such as paclitaxel) and an albumin; and optionally b) an effective amount of a therapeutic agent (such as carboplatin), wherein treatment is based upon the K-ras mutation status at one or more of G12 or G13 of the K-ras amino acid sequence. In some embodiments, the individual has a K-ras mutation. In some embodiments, there is provided a method of treating ovarian cancer (such as metastatic ovarian cancer) in an individual comprising administering to the individual a) an effective amount of a composition comprising nanoparticles comprising a taxane (such as paclitaxel) and an albumin; and optionally b) an effective amount of a therapeutic agent (such as bevacizumab), wherein treatment is based upon the individual having a mutation at one or more of G12 or G13 of the K-ras amino acid sequence. In some embodiments, there is provided a method of treating ovarian cancer (such as metastatic ovarian cancer) in an individual comprising: a) selecting the individual for treatment on the basis that the individual having a mutation at one or more of G12 or G13 of the K-ras amino acid sequence; and b) administering to the individual i) an effective amount of a composition comprising nanoparticles comprising a taxane (such as paclitaxel) and an albumin; and optionally, ii) an effective amount of a therapeutic agent (such as bevacizumab). In some embodiments, there is provided a method of treating ovarian cancer (such as metastatic ovarian cancer) in an individual comprising: a) assessing the K-ras mutation status of the individual; b) selecting the individual for treatment on the basis that the individual having a mutation at one or more of G12 or G13 of the K-ras amino acid sequence; and c) administering to the individual i) an effective amount of a composition comprising nanoparticles comprising a taxane (such as paclitaxel) and an albumin; and optionally, ii) an effective amount of a therapeutic agent (such as bevacizumab). In some embodiments, the K-ras mutation is one or more selected from the group consisting of G12C, G12R, G12A, G12D, G12V, and G13D.

Also provided herein are methods of assessing whether an individual with cancer is more likely to respond or less likely to respond to treatment, wherein the treatment comprises i) an effective amount of a composition comprising nanoparticles comprising a taxane and an albumin and ii) an effective amount of a therapeutic agent, said method comprising determining the K-ras mutation status in the individual, wherein a K-ras mutation indicates that the individual is more likely to respond, and wherein a wild-type K-ras indicates that the individual is less likely to respond to the treatment. In some embodiments, the method further comprises administering to the individual i) an effective amount of a composition comprising nanoparticles comprising a taxane and an albumin, and/or ii) an effective amount of a therapeutic agent to the individual who is determined to be likely to respond to the treatment. In some embodiments, the amount of the therapeutic agent is determined based upon the K-ras mutation status. In some embodiments, the amount of the composition comprising nanoparticles comprising a taxane and an albumin is determined based upon the K-ras mutation status.

In some embodiments, a K-ras mutation compared to a reference indicates that a) the individual is more likely to respond to treatment or b) the individual is selected for treatment. In some embodiments, a wild-type K-ras compared to a reference indicates that a) the individual is less likely to respond to treatment or b) the individual is not selected for treatment.

The K-ras mutation status in an individual can be determined by analyzing a sample from the individual. Suitable samples include, but are not limited to, tumor tissue, normal tissue adjacent to the tumor, normal tissue distal to the tumor, or peripheral blood lymphocytes. In some embodiments, the sample is a tumor tissue. In some embodiments, the sample is a biopsy containing cancer cells, such as fine needle aspiration of cancer cells (e.g., pancreatic cancer cells) or laparoscopy obtained cancer cells (e.g., pancreatic cancer cells). In some embodiments, the biopsied cells are centrifuged into a pellet, fixed, and embedded in paraffin prior to the analysis. In some embodiments, the biopsied cells are flash frozen prior to the analysis. In some embodiments, the biopsied cells are mixed with an antibody that recognizes the K-ras protein or fragment thereof. In some embodiments, the biopsied cells are mixed with a nucleic acid that recognizes the K-ras gene or fragment thereof.

In some embodiments, the sample comprises a circulating metastatic cancer cell. In some embodiments, the sample is obtained by sorting circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis. In some embodiments, the CTCs are pancreatic cancer cells. In some embodiments, the CTCs are colorectal cancer cells. In some embodiments, the CTCs are non-small cell lung carcinoma cells.

In some embodiments of any of the methods, the taxane in the nanoparticles are coated with albumin.

In some embodiments of any of the methods, the composition comprising nanoparticles comprising taxane and albumin is substantially free of surfactant.

In some embodiments of any of the methods, the treatment comprises administration of the composition comprising nanoparticles comprising the taxane and the albumin over less than about 30 minutes. In some embodiments of any of the methods, the treatment comprises a dose of the composition comprising nanoparticles comprising the taxane and the albumin between about 50 mg/m² and about 125 mg/m². In some embodiments, the dose of the composition comprising nanoparticles comprising the taxane and the albumin is about 50 mg/m², about 75 mg/m², or about 100 mg/m². In some embodiments of any of the methods, the treatment comprises administration of the composition comprising nanoparticles comprising the taxane and the albumin parenterally. In some embodiments of any of the methods, the treatment comprises administration of the composition comprising nanoparticles comprising the taxane and the albumin intravenously. In some embodiments of any of the methods, the treatment comprises administration of the composition comprising nanoparticles comprising the taxane and the albumin weekly. In some embodiments of any of the methods, the treatment comprises administration of the composition comprising nanoparticles comprising the taxane and the albumin without any steroid premedication and/or without G-CSF prophylaxis. In some embodiments of any of the methods, the composition comprising nanoparticles comprising a taxane and an albumin and the therapeutic agent are administered sequentially.

In some embodiments of any of the methods, the taxane is paclitaxel.

In some embodiments of any of the methods, the taxane is docetaxel.

In some embodiments of any of the methods, the treatment comprises an amount (dose) of a therapeutic agent between about 5 mg/kg and about 60 mg/kg. In some embodiments, the amount (dose) of a therapeutic agent is about 10 mg/kg, about 20 mg/kg, about 30 mg/kg, about 35 mg/kg, about 40 mg/kg, about 45 mg/kg, or about 50 mg/kg. In some embodiments of any of the methods, the treatment comprises administration of a therapeutic agent orally. In some embodiments of any of the methods, the treatment comprises administration of a therapeutic agent weekly.

In some embodiments of any of the methods, the therapeutic agent is gemcitabine.

In some embodiments of any of the methods, the cancer is selected from the group consisting of biliary tract cancer, bladder cancer, bone cancer, brain cancer, breast cancer, cervical cancer, colorectal cancer, endometrial cancer, eye cancer, genital tract cancer, haematopoietic and lymphoid tissue cancer, kidney cancer, liver cancer, lung cancer, ovarian cancer, pancreatic cancer, prostate cancer, skin cancer, small intestinal cancer, stomach cancer, and thymic cancer. In some embodiments of any of the methods, the method is first-line therapy.

In some embodiments of any of the methods, the cancer is pancreatic cancer. Pancreatic cancers that can be treated with methods described herein include, but are not limited to, exocrine pancreatic cancers and endocrine pancreatic cancers. Exocrine pancreatic cancers include, but are not limited to, adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseuodpapillary tumors, pancreatic ductal carcinomas, and undifferentiated carcinomas. In some embodiments, the exocrine pancreatic cancer is pancreatic ductal carcinoma. Endocrine pancreatic cancers include, but are not limited to, insulinomas and glucagonomas. In some embodiments, the pancreatic cancer is any of early stage pancreatic cancer, non-metastatic pancreatic cancer, primary pancreatic cancer, resected pancreatic cancer, advanced pancreatic cancer, locally advanced pancreatic cancer, metastatic pancreatic cancer, unresectable pancreatic cancer, pancreatic cancer in remission, recurrent pancreatic cancer, pancreatic cancer in an adjuvant setting, or pancreatic cancer in a neoadjuvant setting. In some embodiments, the pancreatic cancer is locally advanced pancreatic cancer, unresectable pancreatic cancer, or metastatic pancreatic ductal carcinoma. In some embodiments, the pancreatic cancer is resistant to the gemcitabine-based therapy. In some embodiments, the pancreatic cancer is refractory to the gemcitabine-based therapy.

In some embodiments, the individual has a pancreatic cancer (such as metastatic cancer). In some embodiments, the individual has locally advanced unresectable pancreatic cancer. In some embodiments, the primary location of the pancreatic cancer is the head of the pancreas. In some embodiments, the primary location of the pancreatic cancer is the body of the pancreas. In some embodiments, the primary location of the pancreatic cancer is the tail of the pancreas. In some embodiments, the individual has metastasis in the liver. In some embodiments, the individual has pulmonary metastasis. In some embodiments, the individual has peritoneal carcinomatosis. In some embodiments, the individual has stage IV pancreatic cancer at the time of diagnosis of pancreatic cancer. In some embodiments, the individual has 3 or more metastatic sites. In some embodiments, the individual has more than 3 metastatic sites. In some embodiments, the individual has a serum CA19-9 level that is ≧59×ULN (Upper Limit of Normal). In some embodiments, the individual has Karnofsky performance status (KPS) of between 70 and 80. In some embodiments, the individual has adenocarcinoma of the pancreas.

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of nab-paclitaxel (for example about 5 mg/ml nab-paclitaxel); and (ii) an effective amount of gemcitabine, wherein the dose of paclitaxel in the nanoparticle composition is about 125 mg/m² on days 1, 8, and 15 of each 28 day cycle, wherein the dose of gemcitabine is about 1000 mg/m² on days 1, 8, and 15 of each 28 day cycle, and wherein the individual is selected for treatment based on the K-ras mutation status. In some embodiments, the gemcitabine is administered immediately after the completion of the administration of the nanoparticle composition.

In some embodiments, there is provided a method of treating locally advanced unresectable or metastatic adenocarcinoma of the pancreas in a human individual comprising intravenously administering (such as by intravenous infusion over about 30 to about 40 minutes) to the individual (i) an effective amount of nab-paclitaxel (for example about 5 mg/ml nab-paclitaxel); and (ii) an effective amount of gemcitabine, wherein the dose of paclitaxel in the nanoparticle composition is about 125 mg/m² on days 1, 8, and 15 of each 28 day cycle, wherein the dose of gemcitabine is about 1000 mg/m² on days 1, 8, and 15 of each 28 day cycle, and wherein the individual has a K-ras mutation (such as K-ras G12 mutation). In some embodiments, the gemcitabine is administered immediately after the completion of the administration of the nanoparticle composition.

Any of the methods described herein can be used for any one or more of the following purposes: alleviating one or more symptoms of cancer, delaying progression of cancer, shrinking cancer tumor size, disrupting (such as destroying) cancer stroma, inhibiting cancer tumor growth, prolonging overall survival, prolonging disease-free survival, prolonging time to cancer disease progression, preventing or delaying cancer tumor metastasis, reducing (such as eradiating) preexisting cancer tumor metastasis, reducing incidence or burden of preexisting cancer tumor metastasis, preventing recurrence of cancer, and/or improving clinical benefit of cancer.

In some embodiments of any of the methods, the nanoparticles in the composition have an average diameter of no greater than about 200 nm. In some embodiments of any of the methods, the albumin is human serum albumin.

In some embodiments of any of the methods, the individual is a human.

K-ras mutations described herein have been identified in several cancers. In some embodiments of any of the methods herein, the K-ras mutation status is determined at one or more positions disclosed in Table 1. In some embodiments, the K-ras mutation status is determined at one position disclosed in Table 1. In some embodiments, the K-ras mutation status is determined at one or more of G12, G13 S17, P34 or Q61 of the K-ras amino acid sequence of SEQ ID NO:2. In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other further embodiments, the K-ras mutation status is determined at one or more of G12, G13, Q61, K117 or A146 of the K-ras amino acid sequence of SEQ ID NO:2. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other further embodiments, K-ras mutation status is determined at one or more of G12, G13 or Q61 of the K-ras amino acid sequence of SEQ ID NO:2. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H.

In any of the embodiments disclosed herein, the K-ras mutation status is determined in exon 2 of the K-ras gene. In any of the embodiments disclosed herein, the K-ras mutation status is determined in exon 3 of the K-ras gene.

In any of the embodiments disclosed herein, the K-ras mutation status is determined as compared to a nucleic acid encoding a K-ras protein. In some embodiments, the nucleic acid sequence encoding a K-ras protein comprises:

(SEQ ID NO: 1)
ATGACTGAATATAAACTTGTGGTAGTTGGAGCTGGTGGCGTAGGCAAGAGTGCCTTGAC
GATACAGCTAATTCAGAATCATTTTGTGGACGAATATGATCCAACAATAGAGGATTCCT
ACAGGAAGCAAGTAGTAATTGATGGAGAAACCTGTCTCTTGGATATTCTCGACACAGCA
GGTCAAGAGGAGTACAGTGCAATGAGGGACCAGTACATGAGGACTGGGGAGGGCTTTC
TTTGTGTATTTGCCATAAATAATACTAAATCATTTGAAGATATTCACCATTATAGAGAAC
AAATTAAAAGAGTTAAGGACTCTGAAGATGTACCTATGGTCCTAGTAGGAAATAAATGT
GATTTGCCTTCTAGAACAGTAGACACAAAACAGGCTCAGGACTTAGCAAGAAGTTATG
GAATTCCTTTTATTGAAACATCAGCAAAGACAAGACAGGGTGTTGATGATGCCTTCTAT
ACATTAGTTCGAGAAATTCGAAAACATAAAGAAAAGATGAGCAAAGATGGTAAAAAGA
AGAAAAAGAAGTCAAAGACAAAGTGTGTAATTATGTAA

In any of the embodiments disclosed herein, the K-ras mutation status is determined as compared to an amino acid sequence encoding a K-ras protein. In some embodiments, the amino acid sequence encoding a K-ras protein comprises:

(SEQ ID NO: 2)
MTEYKLVVVGAGGVGKSALTIQLIQNHFVDEYDPTIEDSYRKQVVIDGETCLLDILDTAGQE
EYSAMRDQYMRTGEGFLCVFAINNTKSFEDIHHYREQIKRVKDSEDVPMVLVGNKCDLPSR
TVDTKQAQDLARSYGIPFIETSAKTRQGVDDAFYTLVREIRKHKEKMSKDGKKKKKKSKTK
CVIM

In some embodiments, an amino acid encoding a K-ras protein is identified by Genbank accession number NP_{—}004976.2. In other embodiments, an amino acid encoding a K-ras protein is identified by Genbank accession number NP_{—}203524.1.

In some embodiments, an mRNA encoding a K-ras protein is identified by Genbank accession number NM_{—}004985.3. In other embodiments, an mRNA encoding a K-ras protein is identified by Genbank accession number NM_{—}033360.2.

In any of the embodiments disclosed herein, the K-ras mutation status is determined in exon 2 of the K-ras gene.

In any of the embodiments disclosed herein, the K-ras mutation status is determined in exon 3 of the K-ras gene.

In any of the embodiments disclosed herein, the K-ras mutations include silent mutations,

In any of the embodiments disclosed herein, evaluated K-ras mutations include, but are not limited to, the K-ras gene mutations and/or K-ras protein mutations described in Table 1. In any of the embodiments disclosed herein, the K-ras mutation status is determined at any of the K-ras gene positions and/or K-ras protein positions described in Table 1. The present application encompasses treatments and methods based on any one or more of the mutations disclosed herein.

In any of the embodiments disclosed herein, the K-ras mutations include silent mutations (such as G12G).

TABLE 1
K-ras mutations in different types of cancer

| | Kras gene | KRAS protein |
|---|---|---|
| Cancer Type | mutations | mutations |
| Biliary tract cancer | c.34G > T | G12C |
| | c.34G > A | G12S |
| | c.34G > C | G12R |
| | c.35G > C | G12A |
| | c.35G > A | G12D |
| | c.35G > T | G12V |
| | c.37G > T | G13C |
| | c.37G > A | G13S |
| | c.37G > C | G13R |
| | c.37 38GG > AA | G13N |
| | c.38G > A | G13D |
| | c.38 39GC > AT | G13D |
| | N/A | I24F |
| | c.182A > T | Q61L |
| | c.183A > C | Q61H |
| | c.183A > T | Q61H |
| Bladder cancer | c.34G > T | G12C |
| | c.34G > A | G12S |
| | c.34G > C | G12R |
| | c.35G > C | G12A |
| | c.35G > A | G12D |
| | c.35G > T | G12V |
| | c.37G > C | G13R |
| | c.38G > A | G13D |
| | c.175G > A | A59T |
| | c.181C > G | Q61E |
| | c.181C > A | Q61K |
| | c.182A > T | Q61L |
| | N/A | Q61H |
| Bone cancer | c.35G > C | G12A |
| | c.38G > A | G13D |
| Brain cancer | c.34G > T | G12C |
| | c.35G > C | G12A |
| | c.35G > A | G12D |
| | c.35G > T | G12V |
| | c.181C > G | Q61E |
| | c.182A > G | Q61R |
| | N/A | E63K |
| Breast cancer | c.34G > T | G12C |
| | c.34G > C | G12R |
| | c.35G > C | G12A |
| | c.35G > A | G12D |
| | c.35G > T | G12V |
| | c.38G > A | G13D |
| | c.182A > T | Q61L |
| Cervical cancer | c.34G > T | G12C |
| | c.34G > C | G12R |
| | c.34G > A | G12S |
| | c.35G > C | G12A |
| | c.35G > A | G12D |
| | c.35G > T | G12V |
| | c.38G > A | G13D |
| | c.182A > G | Q61R |
| | c.182A > C | Q61P |
| | c.183A > T | Q61H |
| Colorectal cancer | c.34G > T | G12C |
| | c.34G > C | G12R |
| | c.34G > A | G12S |
| | c.35G > C | G12A |
| | c.35G > A | G12D |
| | c.35G > T | G12V |
| | c.37G > T | G13C |
| | c.37G > C | G13R |
| | c.37G > A | G13S |
| | c.38G > C | G13A |
| | c.38G > A | G13D |
| | c.38G > T | G13V |
| | c.181C > A | Q61K |
| | c.182A > T | Q61L |
| | c.182A > G | Q61R |
| | c.183A > C | Q61H |
| | c.183A > T | Q61H |
| | c.351A > C | K117N |
| | c.351A > T | K117N |
| | c.436G > C | A146P |
| | c.436G > A | A146T |
| | c.437C > T | A146V |
| Endometrial cancer | c.34G > T | G12C |
| | c.34G > C | G12R |
| | c.34G > A | G12S |
| | c.35G > C | G12A |
| | c.35G > A | G12D |
| | c.35G > T | G12V |
| | c.37G > T | G13C |
| | c.37G > A | G13S |
| | c.38G > A | G13D |
| | c.38 39GC > TT | G13V |
| | c.38G > C | G13A |
| | c.38G > T | G13V |
| | c.59C > G | T20R |
| | c.71T > A | I24N |
| | c.176C > A | A59E |
| | c.182A > T | Q61L |
| | c.183A > C | G61H |
| | c.186G > T | E62D |
| Eye cancer | c.35G > A | G12D |
| | c.35G > T | G12V |
| Genital tract cancer | c.38G > A | G13D |
| Haematopoietic and lymphoid | c.13A > G | K5E |
| tissue cancer | c.27 28insGTA | V9 G10insV |
| | c.30 31insGGA | G10 A11insG |
| | c.32C > T | A11V |
| | c.34G > T | G12C |
| | c.34G > C | G12R |
| | c.34G > A | G12S |
| | c.35G > C | G12A |
| | c.35G > A | G12D |
| | c.35G > T | G12V |
| | c.35G > A | G12D |
| | c.35G > T | G12V |
| | c.36 37insGGT | G12 G13insG |
| | c.37 37insGCG | G12 G13insA |
| | c.37G > A | G13S |
| | c.37G > T | G13C |
| | c.38G > A | G13D |
| | c.40G > A | V14I |
| | c.44G > A | G15D |
| | c.53C > A | A18D |
| | c.57G > C | L19F |
| | c.57G > T | L19F |
| | c.58A > T | T20S |
| | c.64C > A | Q22K |
| | c.68T > G | L23R |
| | c.80A > T | H27L |
| | c.104C > T | T35I |
| | c.173C > T | T58I |
| | c.175G > A | A59T |
| | c.176C > A | A59E |
| | c.179G > A | G60D |
| | c.181C > A | Q61K |
| | c.182A > T | Q61L |
| | c.182A > C | Q61P |
| | c.182A > G | Q61R |
| | c.183A > T | Q61H |
| | c.183A > C | Q61H |
| | c.186 194del9 | E62 S65 > D |
| | c.199A > C | M67L |
| | c.216G > A | M72I |
| | c.220A > C | T74P |
| | c.351A > T | K117N |
| | c.436G > A | A146T |
| | c.436G > C | A146P |
| | c.437C > T | A146V |
| Kidney cancer | c.34G > T | G12C |
| | c.35G > C | G12A |
| | c.35G > A | G12D |
| | c.176C > G | A59G |
| | c.181C > A | Q61K |
| | c.182A > T | Q61L |
| | c.183A > C | Q61H |
| Liver cancer | c.34G > T | G12C |
| | c.34G > A | G12S |
| | c.35G > A | G12D |
| | c.35G > T | G12V |
| | c.35G > C | G12A |
| | c.37G > T | G13C |
| | c.38 39GC > AT | G13D |
| | c.183A > T | Q61H |
| Non-small cell lung cancer | c.34G > T | G12C |
| | c.34G > C | G12R |
| | c.34G > A | G12S |
| | c.35G > C | G12A |
| | c.35G > A | G12D |
| | c.35G > T | G12V |
| | c.37G > T | G13C |
| | c.37G > C | G13R |
| | c.37G > A | G13S |
| | c.38G > C | G13A |
| | c.38G > A | G13D |
| | c.181C > A | Q61K |
| | c.182A > T | Q61L |
| | c.182A > G | Q61R |
| | c.183A > C | Q61H |
| | c.183A > T | Q61H |
| Neuroblastoma | c.34G > T | G12C |
| | c.35G > C | G12A |
| | c.35G > T | G12V |
| Ovarian cancer | c.34G > T | G12C |
| | c.34G > C | G12R |
| | c.35G > C | G12A |
| | c.35G > A | G12D |
| | c.35G > T | G12V |
| | c.38G > A | G13D |
| Pancreatic cancer | c.34G > T | G12C |
| | c.34G > A | G12S |
| | c.34G > C | G12R |
| | c.34 35GG > TT | G12F |
| | c.34 35GG > CT | G12L |
| | c.34 35GG > AA | G12N |
| | c.35G > C | G12A |
| | c.35G > A | G12D |
| | c.35G > T | G12V |
| | c.37G > T | G13C |
| | c.37G > A | G13S |
| | c.38G > A | G13D |
| | c.38 39GC > AT | G13D |
| | c.38 39GC > TT | G13V |
| | N/A | G13P |
| | c.49A > G | S17G |
| | c.100C > T | P34S |
| | c.181C > A | Q61K |
| | c.182A > T | Q61L |
| | c.182A > G | Q61R |
| | c.183A > C | Q61H |
| | c.183A > T | Q61H |
| Prostate cancer | c.34G > T | G12C |
| | c.34G > C | G12R |
| | c.34G > A | G12S |
| | c.35G > C | G12A |
| | c.35G > A | G12D |
| | c.35G > T | G12V |
| | c.37G > A | G13S |
| | c.38G > A | G13D |
| | c.181C > A | Q61K |
| | c.182A > G | Q61R |
| Skin cancer | c.34G > T | G12C |
| | c.34G > C | G12R |
| | c.34G > A | G12S |
| | c.35G > A | G12D |
| | c.35G > T | G12V |
| | c.38G > A | G13D |
| | c.181C > A | Q61K |
| | c.182A > G | Q61R |
| | c.182A > T | Q61L |
| | c.183A > C | Q61H |
| | c.466T > C | F156L |
| Small Intestine | c.34G > T | G12C |
| | c.34G > A | G12S |
| | c.35G > C | G12A |
| | c.35G > A | G12D |
| | c.35G > T | G12V |
| | N/A | G12N |
| | N/A | G13N |
| | c.38G > A | G13D |
| | c.38G > T | G13V |
| | c.39 40insGGC | G13 V14insG |
| | c.64C > A | Q22K |
| | c.183A > C | Q61H |
| Stomach cancer | c.15A > C | K5N |
| | c.31G > C | A11P |
| | c.32C > T | A11V |
| | c.34G > T | G12C |
| | c.34G > A | G12S |
| | c.34G > C | G12R |
| | c.35G > C | G12A |
| | c.35G > A | G12D |
| | c.35G > T | G12V |
| | c.37G > T | G13C |
| | c.37G > A | G13S |
| | c.38G > A | G13D |
| | c.38G > T | G13V |
| | N/A | L19F |
| | c.175G > A | A59T |
| | c.181C > A | Q61K |
| | c.182A > T | Q61L |
| | c.182A > G | Q61R |
| | c.183A > T | Q61H |
| Thymic cancer | c.35G > C | G12A |
| | c.35G > T | G12V |
| | c.37G > A | G13S |
| | c.43G > A | G15S |

The K-ras mutation status can be determined by methods known in the art. See, for example, Chang et al., BMC Cancer, 2009, 9:179 and Gonzalez de Castro et al., Br J Cancer, 2012, 102(2)345-51. In some embodiments, the K-ras mutation status is determined by sequence analysis, for example sequencing analysis of the genomic DNA or RNA (cDNA) obtained from the individual. The K-ras mutation status may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. Bodily fluids include, but are not limited to, blood, lymph, saliva, semen, peritoneal fluid, cerebrospinal fluid, breast milk, and pleural effusion. In some embodiments, the sample is a blood sample which includes, for example, platelets, lymphocytes, polymorphonuclear cells, macrophages, and erythrocytes.

In some embodiments, the sample is a tumor tissue, normal tissue adjacent to said tumor, normal tissue distal to said tumor, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing cancer cells. In a further embodiment, the biopsy is fine needle aspiration of pancreatic cancer cells. In a further embodiment, the biopsy is laparoscopy obtained pancreatic cancer cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes the K-ras mutation. In some embodiments, a biopsy is taken to determine whether an individual has cancer and is then used as a sample. In some embodiments, the sample is surgically obtained tumor cells. In some embodiments, samples may be obtained at different times than when the determining of the K-ras mutation status occurs.

In some embodiments, the sample comprises a circulating metastatic cancer cell. In some embodiments, the circulating metastatic cancer cell is a pancreatic cancer cell, a colorectal cancer cell, or a non-small lung cancer cell. In some embodiments, the sample is obtained by sorting circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis. In some embodiments, the CTCs are pancreatic cancer cells. In some embodiments, the CTCs are colorectal cancer cells. In some embodiments, the CTCs are non-small cell lung carcinoma cells.

In some embodiments, the K-ras mutation status is determined. In some embodiments, the K-ras mutation status at two or more positions of K-ras are determined; for example, one or more K-ras mutation status at two or more positions selected from the group consisting of G12, G13 S17, P34 or Q61 of the K-ras amino acid sequence of SEQ ID NO:2 can be determined. In some embodiments, one or more K-ras mutation status at two or more positions selected from the group consisting of G12, G13, Q61, K117 or A146 of the K-ras amino acid sequence of SEQ ID NO:2 can be determined. In some embodiments, one or more K-ras mutation status at two or more positions selected from the group consisting of G12, G13 or Q61 of the K-ras amino acid sequence of SEQ ID NO:2 can be determined. The one or more K-ras mutation status positions include, for example, at least two or more K-ras mutation status positions, at least three or more K-ras mutation status positions, at least four or more K-ras mutation status positions, at least five or more K-ras mutation status positions, or at least six or more K-ras mutation status positions.

To practice this method, for example, the sample is an individual's sample containing the tumor tissue, normal tissue adjacent to said tumor, normal tissue distal to said tumor or peripheral blood lymphocytes. Sample nucleic acid for use in the above-described methods can be obtained from any cell type or tissue of a subject. For example, a subject's bodily fluid (e.g. blood) can be obtained by known techniques (e.g., venipuncture). Alternatively, tests can be performed on dry samples (e.g., hair or skin). The samples may be fresh or frozen. In some embodiments, the sample is fixed and embedded in paraffin or the like.

In some embodiments, the method comprises isolating a sample containing the genetic material to be tested. In some embodiments, the methods comprise determining the K-ras mutation status in situ. Accordingly, the methods of this application are not to be limited to requiring isolation of the genetic material prior to analysis.

In some embodiments of any of the methods, the one or more characteristics of cancer further comprise differential levels of SPARC. SPARC (Secreted Protein, Acidic and Rich in Cysteine) is a matricellular protein upregulated in several aggressive cancers. See Porter et al., J. Histochem. Cytochem. 1995; 43:791. The human SPARC gene encodes a 303 amino acid SPARC proteins, while mature SPARC is a 285 amino acid glycoprotein. After cleavage of the signal sequence a 32-kD secreted form is produced which migrates at 43 kD on SDA-PAGE because of glycosylation. In some embodiments, differential levels are determined in tumor tissue, normal tissue adjacent to said tumor, normal tissue distal to said tumor or peripheral blood lymphocytes. In some embodiments, the drug uptake capability is based on the level of SPARC on the tumor stroma.

Methods for measuring gene expression and/or determining sequence for detection of polymorphism are well known in the art and include, but are not limited to, immunological assays, nuclease protection assays, northern blots, in situ hybridization, ELISA, reverse transcriptase Polymerase Chain Reaction (RT-PCR), Real-Time Polymerase Chain Reaction, expressed sequence tag (EST) sequencing, cDNA microarray hybridization or gene chip analysis, subtractive cloning, Serial Analysis of Gene Expression (SAGE), Massively Parallel Signature Sequencing (MPSS), Sequencing-By-Synthesis (SBS), aptamer-based assays, western blot, enzyme immunoassays, and Luminex Platform utilizing color. See, e.g., in Ausubel et al. eds., 1995, Current Protocols In Molecular Biology, Units 2 (Northern Blotting), 4 (Southern Blotting), 15 (Immunoblotting) and 18 (PCR Analysis). Diagnostic procedures can also be performed in situ directly upon tissue sections (fixed and/or frozen) of individual's tissue obtained from biopsies or resections. In some embodiments, the K-ras mutation status is based on mRNA level. In some embodiments, the K-ras mutation status is determined by SNP analysis of a nucleic acid encoding the K-ras protein. In some embodiments, the K-ras mutation status is determined by the identification of a polymorphism.

Microarray technology utilizes nucleic acid hybridization techniques and computing technology to evaluate the mRNA expression profile of thousands of genes within a single experiment. See, e.g., WO 01/75166 published Oct. 11, 2001; U.S. Pat. No. 5,700,637, U.S. Pat. No. 5,445,934, U.S. Pat. No. 5,807,522, Lockhart, Nat. Biotech., 14:1675-1680 (1996); Cheung, V. G. et al., Nat. Gen. 21(Suppl):15-19 (1999). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). The Affymetrix GeneChip® system (e.g., GeneChip® Human Genome U133 Plus 2.0 array from Affymetrix, Inc. (catalog no. 900470)) is commercially available and may be used for measuring gene expression levels.

Amplification of polynucleotides includes methods such as PCR, ligation amplification (or ligase chain reaction, LCR) and amplification methods. These methods are known and widely practiced in the art.). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified. In some embodiments, expression of one or more genetic markers may be assayed by RT-PCR. In some embodiments, the RT-PCR may be quantitative RT-PCR (qRT-PCR). In some embodiments, the RT-PCR is real-time RT-PCR. In some embodiments, the RT-PCR is quantitative real-time RT-PCR. In some embodiments, the real-time RT-PCR may be performed using TaqMan® chemistry (Applied Biosystems). In some embodiments, the real-time RT-PCR may be performed using TaqMan® chemistry (Applied Biosystems) and the ABI Prism® 7700 Sequence Detection System (Applied Biosystems). See, e.g., Overbergh, L. et al., J. Biomol. Tech. 14(1): 33-43 (2003).

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

In other embodiments of the invention, gene expression is determined by analysis of expressed protein in a cell by use of one or more antibodies specific for one or more epitopes of individual gene products (proteins), or proteolytic fragments thereof, in the cell. The cell can be derived from various sources, as described herein, including but not limited to cell lines, bodily fluids, xenografts and biopsies. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR. In some embodiments, analyzing protein content comprises assessing proteomic patterns, such as by mass spectrometry, chromatography, capillary electrophoresis, immunohistochemistry or 2-D gel electrophoresis. See, e.g., Latterich M. et al. Eur J. Cancer. 44:2737-41 (2008); Conrotto P. Exp Oncol. 30:171-80 (2008). In other embodiments, reverse-phase protein lysate microarrays are used. See Paweletz, C. P., et al., Oncogene 20:1981-1989 (2001).

In some embodiments, there is provided a method of treating cancer (such as pancreatic cancer) in an individual by administering to the individual i) an effective amount of a composition comprising nanoparticles comprising a taxane (such as paclitaxel) and albumin, and optionally ii) an effective amount of a therapeutic agent (such as gemcitabine), wherein the individual is selected for treatment based on the individual having a K-ras mutation (such as K-ras G12 mutation) and an altered (e.g., increased) level of SPARC. In some embodiments, there is provided a method of treating cancer (such as pancreatic cancer) in an individual, comprising: (a) assessing the K-ras mutation status and the SPARC level in the individual; and (b) administering to the individual (i) an effective amount of a composition comprising nanoparticles comprising a taxane (such as paclitaxel) and albumin and ii) an effective amount of a therapeutic agent (such as gemcitabine), wherein the individual is selected for treatment based on the individual having a K-ras mutation (such as K-ras G12 mutation) and an altered (e.g., increased) level of SPARC. In some embodiments, there is provided a method of treating cancer (such as pancreatic cancer) in an individual, comprising: (a) assessing the K-ras mutation status and SPARC level in the individual; (b) selecting (e.g., identifying) the individual for treatment based on the individual having a K-ras mutation (such as K-ras G12 mutation) and an altered (e.g., increased) level of SPARC; and (c) administering to the individual i) an effective amount of a composition comprising nanoparticles comprising a taxane (such as paclitaxel) and albumin, and optionally ii) an effective amount of a therapeutic agent (such as gemcitabine).

### Additional Embodiments

Provided herein are also methods of treating cancer, comprising: (a) selecting an individual having a K-ras mutation; and (b) administering to the selected individual i) an effective amount of a composition comprising nanoparticles comprising a taxane (e.g., paclitaxel) and an albumin and ii) an effective amount of a therapeutic agent (e.g., gemcitabine). In some embodiments, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H.

Methods are also provided herein of assessing whether an individual with cancer will likely respond to treatment, wherein the treatment comprises i) an effective amount of a composition comprising nanoparticles comprising a taxane (e.g., paclitaxel) and an albumin and ii) an effective amount of a therapeutic agent (e.g., gemcitabine), the method comprising assessing the K-ras mutation status, wherein a K-ras mutation indicates that the individual will likely be responsive to the treatment. In some embodiments, the K-ras mutation is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In some embodiments, the method further comprises administering i) an effective amount of a composition comprising nanoparticles comprising a taxane (e.g., paclitaxel) and an albumin and ii) an effective amount of an inhibitor of a therapeutic agent (e.g., gemcitabine).

Methods are also provided herein of aiding assessment of whether an individual with cancer will likely respond to or is suitable for treatment, wherein the treatment comprises i) an effective amount of a composition comprising nanoparticles comprising a taxane (e.g., paclitaxel) and an albumin and ii) an effective amount of a therapeutic agent (e.g., gemcitabine), the method comprising evaluating the K-ras mutation status, wherein a K-ras mutation indicates indicate that the individual will likely be responsive to the treatment. In some embodiments, the K-ras mutation is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In some embodiments, the method further comprises administering i) an effective amount of a composition comprising nanoparticles comprising a taxane (e.g., paclitaxel) and an albumin and ii) an effective amount of an inhibitor of a therapeutic agent (e.g., gemcitabine). In some embodiments, the amount (dose) of the therapeutic agent is determined based upon the K-ras mutation status. In some embodiments, the amount (dose) of the composition comprising nanoparticles comprising a taxane (e.g., paclitaxel) and an albumin is determined based upon the K-ras mutation status.

In addition, methods are provided herein of identifying an individual with cancer likely to respond to treatment comprising a) an effective amount of a composition comprising nanoparticles comprising a taxane (e.g., paclitaxel) and an albumin and b) an effective amount of a therapeutic agent (e.g., gemcitabine), the method comprising: (A) assessing the K-ras mutation status; and (B) identifying the individual having a K-ras mutation. In some embodiments, the K-ras mutation is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In some embodiments, the method further comprises administering i) an effective amount of a composition comprising nanoparticles comprising a taxane (e.g., paclitaxel) and an albumin and ii) an effective amount of an inhibitor of a therapeutic agent (e.g., gemcitabine). In some embodiments, the amount (dose) of the therapeutic agent is determined based upon the K-ras mutation status. In some embodiments, the amount (dose) of the composition comprising nanoparticles comprising a taxane (e.g., paclitaxel) and an albumin is determined based upon the K-ras mutation status.

In addition, methods are provided herein of selecting or not selecting an individual with cancer more likely suitable or less likely suitable for treatment comprising a) an effective amount of a composition comprising nanoparticles comprising a taxane (e.g., paclitaxel) and an albumin and b) an effective amount of a therapeutic agent (e.g., gemcitabine), the method comprising: (A) assessing the K-ras mutation status; and (B) selecting the individual having a K-ras mutation. In some embodiments, the K-ras mutation is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H. In some embodiments, the method further comprises administering i) an effective amount of a composition comprising nanoparticles comprising a taxane (e.g., paclitaxel) and an albumin and ii) an effective amount of an inhibitor of a therapeutic agent (e.g., gemcitabine). In some embodiments, the amount (dose) of the therapeutic agent is determined based upon the K-ras mutation status. In some embodiments, the amount (dose) of the composition comprising nanoparticles comprising a taxane (e.g., paclitaxel) and an albumin is determined based upon the level of the K-ras mutation status.

Methods are also provided herein of selecting or not selecting an individual with cancer more likely suitable or less likely suitable for treatment comprising a) an effective amount of a composition comprising nanoparticles comprising a taxane (e.g., paclitaxel) and an albumin and b) an effective amount of a therapeutic agent (e.g., gemcitabine), the method comprising: (A) assessing the K-ras mutation status in a biological sample (e.g., tissue sample) using PCR; (B) identifying the K-ras mutation status as compared to a control; and (C) selecting or not selecting an individual with cancer more likely suitable or less likely suitable for treatment based on the K-ras mutation status. In some embodiments, an individual with a wild-type K-ras is not administered a treatment comprising a) an effective amount of a composition comprising nanoparticles comprising a taxane and an albumin and b) an effective amount of a therapeutic agent. In some embodiments, an individual with a K-ras mutation is administered a treatment comprising a) an effective amount of a composition comprising nanoparticles comprising a taxane and an albumin and b) an effective amount of a therapeutic agent.

Methods are provided herein of determining whether an individual with cancer is more likely suitable or less likely suitable for treatment comprising a) an effective amount of a composition comprising nanoparticles comprising a taxane (e.g., paclitaxel) and an albumin and b) an effective amount of a therapeutic agent (e.g., gemcitabine), the method comprising: assessing the K-ras mutation status. In some embodiments, the K-ras mutation status is assessed at one or more positions selected from the group consisting of G12, G13 S17, P34 or Q61 of the K-ras amino acid sequence of SEQ ID NO:2. In some embodiments, the K-ras mutation status is assessed at one or more positions selected from the group consisting of G12, G13, Q61, K117 or A146 of the K-ras amino acid sequence of SEQ ID NO:2. In some embodiments, the K-ras mutation status is assessed at one or more positions selected from the group consisting of G12, G13 or Q61 of the K-ras amino acid sequence of SEQ ID NO:2. In some embodiments, the method further comprises administering i) an effective amount of a composition comprising nanoparticles comprising a taxane (e.g., paclitaxel) and an albumin and ii) an effective amount of an inhibitor of a therapeutic agent (e.g., gemcitabine). In some embodiments, the amount (dose) of the therapeutic agent is determined based upon the K-ras mutation status. In some embodiments, the amount (dose) of the composition comprising nanoparticles comprising a taxane (e.g., paclitaxel) and an albumin is determined based upon the level of the K-ras mutation status.

Also provided herein are methods of adjusting therapy treatment of an individual with cancer receiving a) an effective amount of a composition comprising nanoparticles comprising a taxane (e.g., paclitaxel) and an albumin and b) an effective amount of a therapeutic agent (e.g., gemcitabine), the method comprising assessing the K-ras mutation status in a sample isolated from the individual, wherein a wild-type K-ras indicates that the therapy treatment of the individual is adjusted. In some embodiments, the amount (dose) of the therapeutic agent is adjusted. In some embodiments, the amount (dose) of the composition comprising nanoparticles comprising a taxane (e.g., paclitaxel) and an albumin is adjusted.

Also provided herein are methods of adjusting therapy treatment of an individual with cancer receiving a) an effective amount of a composition comprising nanoparticles comprising a taxane (e.g., paclitaxel) and an albumin and b) an effective amount of a therapeutic agent (e.g., gemcitabine), the method comprising assessing the K-ras mutation status in a sample isolated from the individual, wherein a K-ras mutation indicates that the therapy treatment of the individual is adjusted. In some embodiments, the amount (dose) of the therapeutic agent is adjusted. In some embodiments, the amount (dose) of the composition comprising nanoparticles comprising a taxane (e.g., paclitaxel) and an albumin is adjusted.

Provided herein are also methods for marketing a combination therapy comprising a) an effective amount of a composition comprising nanoparticles comprising a taxane (e.g., paclitaxel) and an albumin and b) an effective amount of a therapeutic agent (e.g., gemcitabine) for use in a cancer individual subpopulation, the methods comprising informing a target audience about the use of the combination therapy for treating the individual subpopulation characterized by the individuals of such subpopulation having a sample which has a K-ras mutation. In some embodiments, the K-ras mutation is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the K-ras mutation is one or more selected from the group consisting of G12C, G12R, G12S, G12A, G12D, G12V, G13C, G13R, G13S, G13A, G13D, Q61K, Q61L, Q61R and Q61H.

In some embodiments of any of the methods, the K-ras mutation status is determined at one or more positions selected from the group consisting of G12, G13 S17, P34 or Q61 of the K-ras amino acid sequence of SEQ ID NO:2. In some embodiments, the K-ras mutation status is determined at one or more positions selected from the group consisting of G12, G13, Q61, K117 or A146 of the K-ras amino acid sequence of SEQ ID NO:2. In some embodiments, the K-ras mutation status is determined at one or more positions selected from the group consisting of G12, G13 or Q61 of the K-ras amino acid sequence. In some embodiments, the K-ras mutation status is a K-ras mutation at one or more positions selected from the group consisting of G12, G13 S17, P34 or Q61 of the K-ras amino acid sequence as compared to a reference. In some embodiments, the K-ras mutation status is a K-ras mutation at one or more positions selected from the group consisting of G12, G13, Q61, K117 of the K-ras amino acid sequence as compared to a reference. In some embodiments, the K-ras mutation status is a K-ras mutation at one or more positions selected from the group consisting of G12, G13 or Q61 of the K-ras amino acid sequence as compared to a reference. In some embodiments, high expression levels may indicate that the individual is more likely to respond to treatment comprising i) an effective amount of a composition comprising nanoparticles comprising taxane (e.g., paclitaxel) and an albumin and ii) an effective amount of a therapeutic agent (e.g., gemcitabine). Further, an individual may be selected for treatment if the K-ras mutation status is a K-ras mutation selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H compared to a reference. In some embodiments, a K-ras mutation selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V compared to a reference. In some embodiments, a K-ras mutation selected from the group consisting of G12C, G12R, G12S, G12A, G12D, G12V, G13C, G13R, G13S, G13A, G13D, Q61K, Q61L, Q61R and Q61H compared to a reference. Wild-type K-ras at positions selected from the group consisting of G12, G13 S17, P34 or Q61 of the K-ras amino acid sequence as compared to a reference may indicate that the individual is less likely to respond to treatment comprising i) an effective amount of a composition comprising nanoparticles comprising taxane (e.g., paclitaxel) and an albumin and ii) an effective amount of a therapeutic agent (e.g., gemcitabine). In some embodiments, the wild-type K-ras positions selected from the group consisting of G12, G13, Q61, K117 of the K-ras amino acid sequence as compared to a reference. In some embodiments, the wild-type K-ras positions selected from the group consisting of G12, G13 or Q61 of the K-ras amino acid sequence as compared to a reference.

In some embodiments of any of the methods described herein, the composition comprising nanoparticles comprising a taxane (e.g., paclitaxel) and an albumin (such as human serum albumin), wherein the taxane (e.g., paclitaxel) in the nanoparticles is coated with the albumin. In some embodiments, the average particle size of the nanoparticles in the composition is no greater than about 200 nm (such as less than about 200 nm). In some embodiments, the composition comprises Nab-paclitaxel (Abraxane®). In some embodiments, the composition is the Nab-paclitaxel (Abraxane®). In some embodiments, the nanoparticle composition and the therapeutic agent (e.g., gemcitabine) have synergistic effect on treating cancer.

In some embodiments of any of the methods, the taxane is selected from a group consisting of paclitaxel, docetaxel, ortataxel, and protaxel. In some embodiments the taxane is docetaxel. In some embodiments, the taxane is paclitaxel.

Therapeutic agents for the treatment of cancer based upon the K-ras mutations status are provided herein. In some embodiments, the therapeutic agent is a chemotherapeutic agent or an antibody. In some embodiments of any of the methods, the chemotherapeutic therapeutic agent is a hydrophilic nucleoside, a pyrimidine nucleoside, or a deoxycytidine analog. In some embodiments of any of the methods, the chemotherapeutic agent is selected from the group consisting of 5-fluororuracil (e.g., CARAC® or EFUDEX®), gemcitabine (GEMZAR®), pemetrexed (e.g., ALIMTA®), raltitrexed (e.g., TOMUDEX®), and capecitabine (e.g., XELODA®), cladribine, clofarabine, cytarabine, fludarabine, or gemcitabine. In some embodiments of any of the methods, the therapeutic agent is gemcitabine or a derivative thereof. In some embodiments, the therapeutic agent is gemcitabine. Derivatives of gemcitabine include, but are not limited to, compounds that are structurally similar to gemcitabine, or are in the same general chemical class as gemcitabine, analogs of gemcitabine, or pharmaceutically acceptable salts of gemcitabine or its derivatives or analogs. An exemplary gemcitabine derivative includes lipophilic gemcitabine. In some embodiments, the derivative of gemcitabine retains one or more similar biological, pharmacological, chemical and/or physical properties (including, for example, functionality) as gemcitabine. In some embodiments, the therapeutic agent is an antibody (such as, for example, Avastin® or Herceptin®). In some embodiments, the therapeutic agent is Avastin®. In some embodiments, the therapeutic agent is Herceptin®.

In some embodiments, the therapeutic agent is a folinic acid. In some embodiments, the therapeutic agent is leucovorin.

In some embodiments, the therapeutic agent is a topoisomerase inhibitor. In some embodiments, the therapeutic agent is irinotecan.

Combinations of therapeutic agents are provided by the invention herein. In some embodiments, the therapeutic agent is a combination of fluorouracil (5-FU) and leucovorin. In some embodiments, the therapeutic agent is a combination of fluorouracil (5-FU), leucovorin, and oxaliplatin. In some embodiments, the therapeutic agent is a combination of capecitabine and irinotecan. In some embodiments, the therapeutic agent is a combination of capecitabine and oxaliplatin. In some embodiments, the therapeutic agent is a combination of fluorouracil (5-FU) and oxaliplatin. In some embodiments, the therapeutic agent is a combination of fluorouracil (5-FU) and irinotecan. In any of the embodiments, the combination of therapeutic agents is provided for treating colorectal cancer.

In some embodiments, the composition comprising nanoparticles comprising a taxane (e.g., paclitaxel) and an albumin (such as human serum albumin) further comprises a therapeutic agent. In some embodiments, the composition comprising nanoparticles comprising the taxane (e.g., paclitaxel) and the albumin and the therapeutic agent (e.g., gemcitabine) are sequentially administered, concurrently administered, or simultaneously administered.

In some embodiments, the composition comprising nanoparticles comprising the taxane (e.g., paclitaxel) and the albumin is administered without any steroid premedication and/or without G-CSF prophylaxis.

In some embodiments of any of the methods herein, the methods are predictive of and/or result in a measurable reduction in tumor size or evidence of disease or disease progression, complete response, partial response, stable disease, increase or elongation of progression free survival, or increase or elongation of overall survival. In some embodiments of any of the methods above, an individual is likely to respond as evident by a measurable reduction in tumor size or evidence of disease or disease progression, complete response, partial response, stable disease, increase or elongation of progression free survival, increase or elongation of overall survival.

In some embodiments of any of the methods, the method comprises a method of inhibiting cancer cell proliferation (such as tumor growth) in an individual, comprising administering to the individual a) an effective amount of a composition comprising nanoparticles comprising a taxane (e.g., paclitaxel) and an albumin and b) an effective amount of a therapeutic agent (e.g., gemcitabine). In some embodiments, at least about 10% (including for example at least about any of 20%, 30%, 40%, 60%, 70%, 80%, 90%, or 100%) cell proliferation is inhibited.

In some embodiments of any of the methods, the method comprises a method of inhibiting tumor metastasis in an individual, comprising administering to the individual a) an effective amount of a composition comprising nanoparticles comprising a taxane (e.g., paclitaxel) and an albumin and b) an effective amount of a therapeutic agent (e.g., gemcitabine). In some embodiments, at least about 10% (including for example at least about any of 20%, 30%, 40%, 60%, 70%, 80%, 90%, or 100%) metastasis is inhibited. In some embodiments, method of inhibiting metastasis to lymph node is provided.

In some embodiments of any of the methods, the method comprises a method of reducing tumor size in an individual, comprising administering to the individual a) an effective amount of a composition comprising nanoparticles comprising a taxane (e.g., paclitaxel) and an albumin and/or b) an effective amount of a therapeutic agent (e.g., gemcitabine). In some embodiments, the tumor size is reduced at least about 10% (including for example at least about any of 20%, 30%, 40%, 60%, 70%, 80%, 90%, or 100%).

In some embodiments of any of the methods, the method comprises a method of prolonging progression-free survival of cancer in an individual, comprising administering to the individual a) an effective amount of a composition comprising nanoparticles comprising a taxane (e.g., paclitaxel) and an albumin and/or b) an effective amount of a therapeutic agent (e.g., gemcitabine). In some embodiments, the method prolongs the time to disease progression by at least any of 1, 2, 3, 4, 5, 6, 7, 8, 9, 10, 11, or 12 weeks.

In some embodiments of any of the methods, the method comprises a method of prolonging survival of an individual having cancer, comprising administering to the individual a) an effective amount of a composition comprising nanoparticles comprising a taxane (e.g., paclitaxel) and an albumin and/or b) an effective amount of a therapeutic agent (e.g., gemcitabine). In some embodiments, the method prolongs the survival of the individual by at least any of 1, 2, 3, 4, 5, 6, 7, 8, 9, 10, 11, 12, 18, or 24 month.

In some embodiments of any of the methods, the method comprises a method of reducing AEs and SAEs in an individual having cancer, comprising administering to the individual a) a composition comprising nanoparticles comprising a taxane (e.g., paclitaxel) and an albumin and/or b) a therapeutic agent (e.g., gemcitabine) compared to administering to the individual a) Taxol® and/or b) of a therapeutic agent (e.g., gemcitabine).

In some embodiments of any of the methods described herein, the method is predictive of and/or results in an objective response (such as a partial response or complete response).

In some embodiments of any of the methods described herein, the method is predictive of and/or results in improved quality of life.

In some embodiments, a lower amount of each pharmaceutically active compound is used as part of a combination therapy compared to the amount generally used for individual therapy. In some embodiments, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. For example, the use of a small amount of pharmaceutically active compound may result in a reduction in the number, severity, frequency, or duration of one or more side-effects associated with the compound.

The methods described herein can be used for and/or predictive of any one or more of the following purposes: alleviating one or more symptoms of cancer, delaying progressing of cancer, shrinking tumor size, inhibiting tumor growth, prolonging overall survival, prolonging progression free survival, preventing or delaying tumor metastasis, reducing (such as eradiating) preexisting tumor metastasis, reducing incidence or burden of preexisting tumor metastasis, or preventing recurrence.

### Exemplary Additional Embodiments

The present application in some embodiments provides a method of treating cancer in an individual comprising administering to the individual an effective amount of a composition comprising nanoparticles comprising a taxane and an albumin, wherein the individual has a K-ras mutation.

The present application in some embodiments provides a method of treating cancer in an individual comprising administering to the individual an effective amount of a composition comprising nanoparticles comprising a taxane and an albumin, wherein the K-ras mutation status is used as a basis for selecting the individual for treatment.

In some embodiments according to (or as applied to) any of the embodiments above, the individual is selected for treatment if the individual has a K-ras mutation.

In some embodiments according to (or as applied to) any of the embodiments above, the K-ras mutation is at one or more of G12, G13, S17, P34 or Q61 of the K-ras amino acid sequence.

In some embodiments according to (or as applied to) any of the embodiments above, the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H.

In some embodiments according to (or as applied to) any of the embodiments above, the K-ras mutation is at one or more of G12, G13, Q61, K117 or A146 of the K-ras amino acid sequence.

In some embodiments according to (or as applied to) any of the embodiments above, the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V.

In some embodiments according to (or as applied to) any of the embodiments above, the K-ras mutation is at one or more of G12, G13 or Q61 of the K-ras amino acid sequence.

In some embodiments according to (or as applied to) any of the embodiments above, the K-ras mutation is one or more selected from the group consisting of G12C, G12R, G12S, G12A, G12D, G12V, G13C, G13R, G13S, G13A, G13D, Q61K, Q61L, Q61R and Q61H.

In some embodiments according to (or as applied to) any of the embodiments above, the method further comprises administering to the individual an effective amount of a therapeutic agent.

In some embodiments according to (or as applied to) any of the embodiments above, the therapeutic agent is a chemotherapeutic agent or an antibody.

In some embodiments according to (or as applied to) any of the embodiments above, the chemotherapeutic agent is gemcitabine.

In some embodiments according to (or as applied to) any of the embodiments above, the method comprises determining the K-ras mutation status of the individual prior to administering to the individual an effective amount of the composition comprising nanoparticles comprising a taxane and an albumin.

In some embodiments according to (or as applied to) any of the embodiments above, the composition comprising nanoparticles comprising a taxane and an albumin is administered intravenously.

In some embodiments according to (or as applied to) any of the embodiments above, the taxane is paclitaxel.

In some embodiments according to (or as applied to) any of the embodiments above, the composition comprising nanoparticles comprising a taxane and albumin and the therapeutic agent are administered sequentially.

In some embodiments according to (or as applied to) any of the embodiments above, the nanoparticles in the composition comprises the taxane coated with the albumin.

In some embodiments according to (or as applied to) any of the embodiments above, the nanoparticles in the composition have an average diameter of less than about 200 nm.

In some embodiments according to (or as applied to) any of the embodiments above, the albumin is human serum albumin.

In some embodiments according to (or as applied to) any of the embodiments above, the individual is human.

The present application in some embodiments provides a kit comprising 1) a composition comprising nanoparticles comprising a taxane and an albumin, and 2) an agent for determining the K-ras mutation status.

In some embodiments according to (or as applied to) any of the embodiments above, the K-ras mutation status is assessed at one or more of G12, G13, S17, P34 or Q61 of the K-ras amino acid sequence.

In some embodiments according to (or as applied to) any of the embodiments above, the K-ras mutation status is assessed at one or more of G12, G13, Q61, K117 or A146 of the K-ras amino acid sequence.

In some embodiments according to (or as applied to) any of the embodiments above, the K-ras mutation status is assessed at one or more of G12, G13 or Q61 of the K-ras amino acid sequence.

In some embodiments according to (or as applied to) any of the embodiments above, the agent for determining the K-ras mutation status is a nucleic acid recognizing the K-ras mutation.

In some embodiments according to (or as applied to) any of the embodiments above, the taxane is paclitaxel.

### Cancers for Treatment

Cancers discussed herein include, but are not limited to, adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, melanoma, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), and Meigs' syndrome.

In some embodiments of any of the methods, the cancer is selected from the group consisting of lung cancer (e.g., NCSLC or SCLC), uterine cancer (e.g., leiomyosarcoma), kidney cancer, ovarian cancer, breast cancer, endometrial cancer, head & neck cancer, pancreatic cancer, and melanoma.

In some embodiments of any of the methods, the cancer is a lymphoid neoplasm (e.g., lymphoma).

In some embodiments, the lymphoid neoplasm (e.g., lymphoma) is a B-cell neoplasm. Examples of B-cell neoplasms include, but are not limited to, precursor B-cell neoplasms (e.g., precursor B-lymphoblastic leukemia/lymphoma) and peripheral B-cell neoplasms (e.g., B-cell chronic lymphocytic leukemia/prolymphocytic leukemia/small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type+/− monocytoid B cells) and/or Nodal (e.g., +/− monocytoid B cells)), splenic marginal zone lymphoma (e.g., +/− villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments, the lymphoid neoplasm (e.g., lymphoma) is a T-cell and/or putative NK-cell neoplasm. Examples of T-cell and/or putative NK-cell neoplasms include, but are not limited to, precursor T-cell neoplasm (precursor T-lymphoblastic lymphoma/leukemia) and peripheral T-cell and NK-cell neoplasms (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic γδ T-cell lymphoma, and subcutaneous panniculitic T-cell lymphoma), angioimmunoblastic T-cell lymphoma (AILD), angiocentric lymphoma, intestinal T-cell lymphoma (e.g., +/− enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's like).

In some embodiments, the lymphoid neoplasm (e.g., lymphoma) is Hodgkin's disease. For example, the Hodgkin's disease may be lymphocyte predominance, nodular sclerosis, mixed cellularity, lymphocyte depletion, and/or lymphocyte-rich.

In some embodiments of any of the methods, the cancer is leukemia. In some embodiments, the leukemia is chronic leukemia. Examples of chronic leukemia include, but are not limited to, chronic myelocytic I (granulocytic) leukemia, chronic myelogenous, and chronic lymphocytic leukemia (CLL). In some embodiments, the leukemia is acute leukemia. Examples of acute leukemia include, but are not limited to, acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia).

In some embodiments of any of the methods, the cancer is a liquid tumor or plasmacytoma. Plasmacytoma includes, but is not limited to, myeloma. Myeloma includes, but is not limited to, an extramedullary plasmacytoma, a solitary myeloma, and multiple myeloma. In some embodiments, the plasmacytoma is multiple myeloma.

In some embodiments of any of the methods, the cancer is multiple myeloma. Examples of multiple myeloma include, but are not limited to, IgG multiple myeloma, IgA multiple myeloma, IgD multiple myeloma, IgE multiple myeloma, and nonsecretory multiple myeloma. In some embodiments, the multiple myeloma is IgG multiple myeloma. In some embodiments, the multiple myeloma is IgA multiple myeloma. In some embodiments, the multiple myeloma is a smoldering or indolent multiple myeloma. In some embodiments, the multiple myeloma is progressive multiple myeloma.

In some embodiments of any of the methods, the cancer is a solid tumor. In some embodiments, the solid tumor includes, but is not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Kaposi's sarcoma, soft tissue sarcoma, uterine sacronomasynovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In some embodiments of any of the methods, the cancer is breast cancer. In some embodiments, the breast cancer is early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some specific embodiments, the breast cancer is in a neoadjuvant setting. In some embodiments, there are provided methods of treating cancer at advanced stage(s).

In some embodiments of any of the methods, the cancer is a renal cell carcinoma (also called kidney cancer, renal adenocarcinoma, or hypernephroma). In some embodiments, the renal cell carcinoma is an adenocarcinoma. In some embodiments, the renal cell carcinoma is a clear cell renal cell carcinoma, papillary renal cell carcinoma (also called chromophilic renal cell carcinoma), chromophobe renal cell carcinoma, collecting duct renal cell carcinoma, granular renal cell carcinoma, mixed granular renal cell carcinoma, renal angiomyolipomas, or spindle renal cell carcinoma. In some embodiments, the renal cell carcinoma is associated with (1) von Hippel-Lindau (VHL) syndrome, (2) hereditary papillary renal carcinoma (HPRC), (3) familial renal oncocytoma (FRO) associated with Birt-Hogg-Dube syndrome (BHDS), or (4) hereditary renal carcinoma (HRC). There are provided methods of treating renal cell carcinoma at any of the four stages, I, II, III, or IV, according to the American Joint Committee on Cancer (AJCC) staging groups. In some embodiments, the renal cell carcinoma is stage IV renal cell carcinoma.

In some embodiments of any of the methods, the cancer is prostate cancer. In some embodiments, the prostate cancer is an adenocarcinoma. In some embodiments, the prostate cancer is a sarcoma, neuroendocrine tumor, small cell cancer, ductal cancer, or a lymphoma. There are provided methods of treating prostate cancer at any of the four stages, A, B, C, or D, according to the Jewett staging system. In some embodiments, the prostate cancer is stage A prostate cancer (The cancer cannot be felt during a rectal exam.). In some embodiments, the prostate cancer is stage B prostate cancer (The tumor involves more tissue within the prostate, it can be felt during a rectal exam, or it is found with a biopsy that is done because of a high PSA level.). In some embodiments, the prostate cancer is stage C prostate cancer (The cancer has spread outside the prostate to nearby tissues.). In some embodiments, the prostate cancer is stage D prostate cancer. In some embodiments, the prostate cancer may be androgen independent prostate cancer (AIPC). In some embodiments, the prostate cancer may be androgen dependent prostate cancer. In some embodiments, the prostate cancer may be refractory to hormone therapy. In some embodiments, the prostate cancer may be substantially refractory to hormone therapy.

In some embodiments of any of the methods, the cancer is lung cancer. In some embodiments, the cancer is lung cancer is a non-small cell lung cancer (NSCLC). Examples of NSCLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC may be, according to TNM classifications, a stage T tumor (primary tumor), a stage N tumor (regional lymph nodes), or a stage M tumor (distant metastasis). In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the cancer is small cell lung cancer (SCLC; also called oat cell carcinoma). The small cell lung cancer may be limited-stage, extensive stage or recurrent small cell lung cancer.

In some embodiments of any of the methods, the cancer is brain cancer. In some embodiments, the brain cancer is glioma, brain stem glioma, cerebellar or cerebral astrocytoma (e.g., pilocytic astrocytoma, diffuse astrocytoma, or anaplastic (malignant) astrocytoma), malignant glioma, ependymoma, oligodenglioma, meningioma, craniopharyngioma, haemangioblastomas, medulloblastoma, supratentorial primitive neuroectodermal tumors, visual pathway and hypothalamic glioma, or glioblastoma. In some embodiments, the brain cancer is glioblastoma (also called glioblastoma multiforme or grade 4 astrocytoma). In some embodiments, the glioblastoma is radiation-resistant. In some embodiments, the glioblastoma is radiation-sensitive. In some embodiments, the glioblastoma may be infratentorial. In some embodiments, the glioblastoma is supratentorial.

In some embodiments of any of the methods, the cancer is melanoma. In some embodiments, the melanoma is cutaneous melanoma. In some embodiments, the melanoma is metastatic melanoma. In some embodiments, the melanoma is metastatic malignant melanoma. In some embodiments, the melanoma is stage IV melanoma (e.g., stage IV cutaneous melanoma). In some embodiments, the metastatic melanoma is at stage M1a. In some embodiments, the metastatic melanoma is at stage M1b. In some embodiments, the metastatic melanoma is at stage M1c. In some embodiments, the individual has not received prior therapy (e.g., prior cytotoxic chemotherapy) for the melanoma (e.g., metastatic melanoma). In some embodiments, the melanoma comprises a mutation in BRAF. In some embodiments, the melanoma does not comprise a mutation in BRAF. In some embodiments, the melanoma is cutaneous melanoma. In some embodiments, the melanoma is melanoma of the skin. In some embodiments, the melanoma is superficial spreading melanoma. In some embodiments, the melanoma is nodular melanoma. In some embodiments, the melanoma is acral lentiginous melanoma. In some embodiments, the melanoma is lentigo maligna melanoma. In some embodiments, the melanoma is mucosal melanoma (e.g., mucosal melanoma in nose, mouth, throat, or genital area). In some embodiments, the melanoma is ocular melanoma. In some embodiments, the melanoma is uveal melanoma. In some embodiments, the melanoma is choroidal melanoma. Melanoma described herein may also be any of the following: cutaneous melanoma, extracutaneous melanoma, superficial spreading melanoma, malignant melanoma, nodular malignant melanoma, nodular melanoma, polypoid melanoma, acral lentiginous melanoma, lentiginous malignant melanoma, amelanotic melanoma, lentigo maligna melanoma, mucosal lentignous melanoma, mucosal melanoma, soft-tissue melanoma, ocular melanoma, desmoplastic melanoma, or metastatic malignant melanoma.

In some embodiments of any of the methods, the cancer is ovarian cancer. In some embodiments, the cancer is ovarian epithelial cancer. Exemplary ovarian epithelial cancer histological classifications include: serous cystomas (e.g., serous benign cystadenomas, serous cystadenomas with proliferating activity of the epithelial cells and nuclear abnormalities but with no infiltrative destructive growth, or serous cystadenocarcinomas), mucinous cystomas (e.g., mucinous benign cystadenomas, mucinous cystadenomas with proliferating activity of the epithelial cells and nuclear abnormalities but with no infiltrative destructive growth, or mucinous cystadenocarcinomas), endometrioid tumors (e.g., endometrioid benign cysts, endometrioid tumors with proliferating activity of the epithelial cells and nuclear abnormalities but with no infiltrative destructive growth, or endometrioid adenocarcinomas), clear cell (mesonephroid) tumors (e.g., begin clear cell tumors, clear cell tumors with proliferating activity of the epithelial cells and nuclear abnormalities but with no infiltrative destructive growth, or clear cell cystadenocarcinomas), unclassified tumors that cannot be allotted to one of the above groups, or other malignant tumors. In various embodiments, the ovarian epithelial cancer is stage I (e.g., stage IA, IB, or IC), stage II (e.g., stage IIA, IIB, or IIC), stage III (e.g., stage IIIA, IIIB, or IIIC), or stage IV.

In some embodiments, the cancer is an ovarian germ cell tumor. Exemplary histologic subtypes include dysgerminomas or other germ cell tumors (e.g., endodermal sinus tumors such as hepatoid or intestinal tumors, embryonal carcinomas, olyembryomas, choriocarcinomas, teratomas, or mixed form tumors). Exemplary teratomas are immature teratomas, mature teratomas, solid teratomas, and cystic teratomas (e.g., dermoid cysts such as mature cystic teratomas, and dermoid cysts with malignant transformation). Some teratomas are monodermal and highly specialized, such as struma ovarii, carcinoid, struma ovarii and carcinoid, or others (e g, malignant neuroectodermal and ependymomas). In some embodiments, the ovarian germ cell tumor is stage I (e.g., stage IA, IB, or IC), stage II (e.g., stage IIA, IIB, or IIC), stage III (e.g., stage IIIA, IIIB, or IIIC), or stage IV.

In some embodiments of any of the methods, the cancer is a pancreatic cancer. In some embodiments, the pancreatic cancer is exocrine pancreatic cancer or endocrine pancreatic cancer. The exocrine pancreatic cancer includes, but is not limited to, adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseuodpapillary tumors, pancreatic ductal carcinomas, and undifferentiated carcinomas. In some embodiments, the exocrine pancreatic cancer is pancreatic ductal carcinoma. The endocrine pancreatic cancer includes, but is not limited to, insulinomas and glucagonomas.

In some embodiments, the pancreatic cancer is early stage pancreatic cancer, non-metastatic pancreatic cancer, primary pancreatic cancer, advanced pancreatic cancer, locally advanced pancreatic cancer, metastatic pancreatic cancer, unresectable pancreatic cancer, pancreatic cancer in remission, or recurrent pancreatic cancer. In some embodiments, the pancreatic cancer is locally advanced pancreatic cancer, unresectable pancreatic cancer, or metastatic pancreatic ductal carcinoma. In some embodiments, the pancreatic cancer is resistant to the gemcitabine-based therapy. In some embodiments, the pancreatic cancer is refractory to the gemcitabine-based therapy. In some embodiments, the pancreatic cancer is resectable (i.e., tumors that are confined to a portion of the pancreas or has spread just beyond it that allows for complete surgical removal), or locally advanced (unresectable) (i.e., the localized tumors may be unresectable because of local vessel impingement or invasion by tumor). In some embodiments, the pancreatic cancer is, according to American Joint Committee on Cancer (AJCC) TNM classifications, a stage 0 tumor (the tumor is confined to the top layers of pancreatic duct cells and has not invaded deeper tissues, and it has not spread outside of the pancreas (e.g., pancreatic carcinoma in situ or pancreatic intraepithelial neoplasia III), a stage IA tumor (the tumor is confined to the pancreas and is less than 2 cm in size, and it has not spread to nearby lymph nodes or distinct sites), a stage IB tumor (the tumor is confined to the pancreas and is larger than 2 cm in size, and it has not spread to nearby lymph nodes or distant sites), a stage IIA tumor (the tumor is growing outside the pancreas but not into large blood vessels, and it has not spread to nearby lymph nodes or distant sites), stage IIB (the tumor is either confined to the pancreas or growing outside the pancreas but not into nearby large blood vessels or major nerves, and it has spread to nearby lymph nodes but not distant sites), stage III (the tumor is growing outside the pancreas into nearby large blood vessels or major nerves, and it may or may not have spread to nearby lymph nodes. It has not spread to distant sites) or stage IV tumor (the cancer has spread to distant sites).

The methods provided herein can be used to treat an individual (e.g., human) who has been diagnosed with pancreatic cancer and has progressed on a prior therapy (e.g., gemcitabine-based, erlotinib-based, or 5-fluorouracil-based therapy). In some embodiments, the individual is resistant to treatment of pancreatic cancer with gemcitabine-based therapy (e.g., gemcitabine monotherapy or gemcitabine combination therapy) and has progressed after treatment (e.g., the pancreatic cancer has been refractory). In some embodiments, the individual is initially responsive to treatment of pancreatic cancer with gemcitabine-based therapy (e.g., gemcitabine monotherapy or gemcitabine combination therapy) but has progressed after treatment. In some embodiments, the individual is non-responsive, less responsive or has stopped responding to treatment with a therapeutic agent (e.g., gemcitabine). In some embodiments, the individual is human. In some embodiments, the individual is at least about any of 30, 35, 40, 45, 50, 55, 60, 65, 70, 75, 80, or 85 years old. In some embodiments, the individual has a family history of pancreatic cancer (e.g., at least 2 first-degree relatives affected with pancreatic cancer without accumulation of other cancers or familial diseases). In some embodiments, the individual has one or more hereditary pancreatic cancer syndromes, including, but not limited to, BRCA2 mutation, familial atypical multiple mole melanoma (FAMMM), peutz-jeghers syndrome, and hereditary pancreatitis. In some embodiments, the individual is a long-time smoker (e.g., more than 10, 15, or 20 years). In some embodiments, the patient has adult-onset diabetes. In some embodiments, the individual is a male. In some embodiments, the individual is a female. In some embodiments, the individual has early stage of pancreatic cancer, non-metastatic pancreatic cancer, primary pancreatic cancer, resected pancreatic cancer, advanced pancreatic cancer, locally advanced pancreatic cancer, metastatic pancreatic cancer, unresectable pancreatic cancer, pancreatic cancer in remission, or recurrent pancreatic cancer. In some embodiments, the individual has Stage 0, IA, IB, IIA, IIB, III, or IV pancreatic cancer according to AJCC (American Joint Commission on Cancer) TNM staging criteria. In some embodiments, the individual has ECOG/WHO/Zubrod score of 0 (asymptomatic), 1 (symptomatic but completely ambulatory), 2 (symptomatic, <50% in bed during the day), 3 (symptomatic, >50% in bed, but not bedbound), or 4 (bedbound). In some embodiments, the individual has a single lesion at presentation. In some embodiments, the individual has multiple lesions at presentation.

In some embodiments, the individual is a human who exhibits one or more symptoms associated with pancreatic cancer. In some embodiments, the individual is at an early stage of pancreatic cancer. In some embodiments, the individual is at an advanced stage of pancreatic cancer. In some embodiments, the individual has non-metastatic pancreatic cancer. In some embodiments, the individual has primary pancreatic cancer. In some of embodiments, the individual is genetically or otherwise predisposed (e.g., having a risk factor) to developing pancreatic cancer. These risk factors include, but are not limited to, age, sex, race, diet, history of previous pancreatic cancer, presence of hereditary pancreatic cancer syndrome (e.g., BRCA2 mutation, familial atypical multiple mole melanoma, Peutz-Jeghers Syndrome, hereditary pancreatitis), genetic (e.g., familial pancreatic cancer) considerations, and environmental exposure. In some embodiments, the individuals at risk for pancreatic cancer include, e.g., those having at least 2 first-degree relatives who have experienced pancreatic cancer without accumulation of other cancers or familial diseases, and those whose risk is determined by analysis of genetic or biochemical markers (e.g., BRCA2, p16, STK11/LKB1, or PRSS1 gene). In some embodiments, the individual is positive for SPARC expression (for example based on IHC standard). In some embodiments, the individual is negative for SPARC expression.

In some embodiments, the individual has a pancreatic cancer (such as metastatic cancer). In some embodiments, the individual has locally advanced unresectable pancreatic cancer. In some embodiments, the primary location of the pancreatic cancer is the head of the pancreas. In some embodiments, the primary location of the pancreatic cancer is the body of the pancreas. In some embodiments, the primary location of the pancreatic cancer is the tail of the pancreas. In some embodiments, the individual has metastasis in the liver. In some embodiments, the individual has pulmonary metastasis. In some embodiments, the individual has peritoneal carcinomatosis. In some embodiments, the individual has stage IV pancreatic cancer at the time of diagnosis of pancreatic cancer. In some embodiments, the individual has 3 or more metastatic sites. In some embodiments, the individual has more than 3 metastatic sites. In some embodiments, the individual has a serum CA19-9 level that is ≧59×ULN (Upper Limit of Normal). In some embodiments, the individual has Karnofsky performance status (KPS) of between 70 and 80. In some embodiments, the individual has adenocarcinoma of the pancreas.

Any of the methods provided herein may be used to treat a primary tumor. Any of the methods of treatment provided herein may also be used to treat a metastatic cancer (that is, cancer that has metastasized from the primary tumor). Any of the methods provided herein may be used to treat cancer at an advanced stage. Any of the methods provided herein may be used to treat cancer at locally advanced stage. Any of the methods provided herein may be used to treat early stage cancer. Any of the methods provided herein may be used to treat cancer in remission. In some of the embodiments of any of the methods provided herein, the cancer has reoccurred after remission. In some embodiments of any of the methods provided herein, the cancer is progressive cancer. Any of the methods t provided herein may be used to treat cancer substantially refractory to hormone therapy. Any of the methods provided herein may be used to treat HER-2 positive cancer. Any of the methods provided herein may be used to treat HER-2 negative cancer. In some embodiments of any of the methods, the cancer is estrogen and progesterone positive. In some embodiments of any of the methods, the cancer is estrogen and progesterone negative.

Any of the methods provided herein may be practiced in an adjuvant setting. Any of the methods provided herein may be practiced in a neoadjuvant setting, i.e., the method may be carried out before the primary/definitive therapy. In some embodiments, any of the methods provided herein may be used to treat an individual who has previously been treated. Any of the methods provided herein may be used to treat an individual who has not previously been treated. Any of the methods provided herein may be used to treat an individual at risk for developing cancer, but has not been diagnosed with cancer. Any of the methods provided herein may be used as a first line therapy. Any of the methods provided herein may be used as a second line therapy.

In some embodiments of any of the methods described herein, the cancer is early stage cancer, non-metastatic cancer, primary cancer, advanced cancer, locally advanced cancer, metastatic cancer, cancer in remission, or recurrent cancer. In some embodiments, the cancer is localized resectable, localized unresectable, or unresectable.

Any of the methods provided herein may be used to treat an individual (e.g., human) who has been diagnosed with or is suspected of having cancer. In some embodiments, the individual may be a human who exhibits one or more symptoms associated with cancer. In some embodiments, the individual may have advanced disease or a lesser extent of disease, such as low tumor burden. In some embodiments, the individual is at an early stage of a cancer. In some embodiments, the individual is at an advanced stage of cancer. In some of the embodiments of any of the methods of treatment provided herein, the individual may be a human who is genetically or otherwise predisposed (e.g., risk factor) to developing cancer who has or has not been diagnosed with cancer. In some embodiments, these risk factors include, but are not limited to, age, sex, race, diet, history of previous disease, presence of precursor disease, genetic (e.g., hereditary) considerations, and environmental exposure (e.g., cigarette, pipe, or cigar smoking, exposure to second-hand smoke, radon, arsenic, asbestos, chromates, chloromethyl ethers, nickel, polycyclic aromatic hydrocarbons, radon progeny, other agents, or air pollution).

In some embodiments of any of the methods described herein, an individual (e.g., human) who has been diagnosed with or is suspected of having cancer can be treated. In some embodiments, the individual is human. In some embodiments, the individual is at least about any of 35, 40, 45, 50, 55, 60, 65, 70, 75, 80, or 85 years old. In some embodiments, the individual is male. In some embodiments, the individual is a female. In some embodiments, the individual has any of the types of cancer described herein. In some embodiments, the individual has a single lesion at presentation. In some embodiments, the individual has multiple lesions at presentation. In some embodiments, the individual is resistant to treatment of cancer with other agents (such as a non-nanoparticle formulation of taxane, e.g., Taxol® or Taxotere®). In some embodiments, the individual is initially responsive to treatment of cancer with other agents (such as a non-nanoparticle formulation of taxane, e.g., Taxol® or Taxotere®) but has progressed after treatment.

### Modes of Administration

The dose of the taxane (such as paclitaxel) nanoparticle compositions and/or the dose of therapeutic agent (such as gemcitabine) administered to an individual (such as a human) according to a method described herein may vary with the particular composition, the mode of administration, and the type of cancer described herein being treated. The dose of the taxane (such as paclitaxel) nanoparticle compositions and/or the dose of therapeutic agent (such as gemcitabine) administered to an individual (such as a human) may also be adjusted (such as reduced) based on an individual's symptoms (such as adverse reactions). In some embodiments, the dose or amount is effective to result in a response. In some embodiments, the dose or amount is effective to result in an objective response (such as a partial response or a complete response). In some embodiments, the dose of the taxane (such as paclitaxel) nanoparticle composition (and/or the dose of therapeutic agent (such as gemcitabine)) administered is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the taxane (such as paclitaxel) nanoparticle composition and/or therapeutic agent (such as gemcitabine). Responses of an individual to the treatment of the methods described herein can be determined using methods known in the field.

In some embodiments, the amount (dose) of the taxane (such as paclitaxel) nanoparticle composition and/or the amount (dose) of therapeutic agent (such as gemcitabine) are sufficient to prolong progression-free survival of the individual. In some embodiments, the amount of the composition (and/or the dose of therapeutic agent (such as gemcitabine)) is sufficient to prolong survival of the individual. In some embodiments, the amount of the composition (and/or the dose of therapeutic agent (such as gemcitabine)) is sufficient to improve quality of life of the individual. In some embodiments, the amount of the composition (and/or the dose of therapeutic agent (such as gemcitabine)) is sufficient to produce clinical benefit of more than about any of 50%, 60%, 70%, or 77% among a population of individuals treated with the taxane (such as paclitaxel) nanoparticle composition and/or therapeutic agent (such as gemcitabine).

In some embodiments, the amount (dose) of the taxane (such as paclitaxel) nanoparticle composition, or therapeutic agent (such as gemcitabine) is an amount sufficient to decrease the size of a pancreatic tumor, decrease the number of tumor cells, or decrease the growth rate of a tumor by at least about any of 10%, 20%, 30%, 40%, 50%, 60%, 70%, 80%, 90%, 95% or 100% compared to the corresponding tumor size, number of pancreatic tumor cells, or tumor growth rate in the same individual prior to treatment or compared to the corresponding activity in other individuals not receiving the treatment. Methods that can be used to measure the magnitude of this effect are known in the field.

In some embodiments, the amount (dose) of the taxane (e.g., paclitaxel) in the composition (and/or therapeutic agent (such as gemcitabine)) is below the level that induces a toxicological effect (i.e., an effect above a clinically acceptable level of toxicity) or is at a level where a potential side effect can be controlled or tolerated when the composition (and/or therapeutic agent (such as gemcitabine)) is administered to the individual.

In some embodiments, the amount of the composition (and/or therapeutic agent (such as gemcitabine)) is close to a maximum tolerated dose (MTD) of the composition (and/or therapeutic agent (such as gemcitabine)) following the same dosing regimen. In some embodiments, the amount of the composition (and/or therapeutic agent (such as gemcitabine)) is more than about any of 80%, 90%, 95%, or 98% of the MTD.

In some embodiments, the amount (dose) of a taxane (e.g., paclitaxel) in the composition is included in any of the following ranges: about 0.1 mg to about 500 mg, about 0.1 mg to about 2.5 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount (dose) of a taxane (e.g., paclitaxel) in the composition (e.g., a unit dosage form) is in the range of about 5 mg to about 500 mg, such as about 30 mg to about 300 mg or about 50 mg to about 200 mg. In some embodiments, the concentration of the taxane (e.g., paclitaxel) in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the taxane (e.g., paclitaxel) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml. In some embodiments, the concentration of the taxane (e.g., paclitaxel) is no more than about any of 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, or 5 mg/ml.

Exemplary amounts (doses) of a taxane (e.g., paclitaxel) in the nanoparticle composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of a taxane (e.g., paclitaxel). In various embodiments, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² of a taxane (e.g., paclitaxel). In some embodiments, the amount (dose) of the taxane (e.g., paclitaxel) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the amount (dose) of a taxane (e.g., paclitaxel) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 100 to about 200 mg/m², about 125 to about 150 mg/m², about 125 to about 175 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the amount (dose) of a taxane (e.g., paclitaxel) in the composition is included in any of the following ranges: about 10 mg/m² to about 400 mg/m², about 25 mg/m² to about 400 mg/m², about 50 mg/m² to about 400 mg/m², about 75 mg/m² to about 350 mg/m², about 75 mg/m² to about 300 mg/m², about 75 mg/m² to about 250 mg/m², about 75 mg/m² to about 200 mg/m², about 75 mg/m² to about 150 mg/m², about 75 mg/m² to about 125 mg/m², about 100 mg/m² to about 260 mg/m², about 100 mg/m² to about 250 mg/m², about 100 mg/m² to about 200 mg/m², or about 125 mg/m² to about 175 mg/m². In some embodiments, the amount (dose) of a taxane (e.g., paclitaxel) in the composition is about 5 to about 300 mg/m², about 100 to about 200 mg/m², about 100 to about 150 mg/m², about 50 to about 150 mg/m², about 75 to about 150 mg/m², about 75 to about 125 mg/m², or about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², about 200 mg/m², about 250 mg/m², about 260 mg/m², or about 300 mg/m².

In some embodiments of any of the above aspects, the amount (dose) of a taxane (e.g., paclitaxel) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In various embodiments, the amount (dose) of a taxane (e.g., paclitaxel) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of a taxane (e.g., paclitaxel).

Exemplary dosing frequencies for the administration of the nanoparticle compositions include, but are not limited to, daily, every two days, every three days, every four days, every five days, every six days, weekly without break, weekly for three out of four weeks, once every three weeks, once every two weeks, or two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the taxane (e.g., paclitaxel) is administered over a period of at least ten days, wherein the interval between each administration is no more than about two days, and wherein the dose of the taxane (e.g., paclitaxel) at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², about 25 mg/m² to about 50 mg/m², or about 50 mg/m² to about 100 mg/m².

The administration of the composition can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months.

In some embodiments, the dosage of a taxane (e.g., paclitaxel) in a nanoparticle composition can be in the range of 5-400 mg/m² when given on a 3 week schedule, or 5-250 mg/m² (such as 75-200 mg/m², 100-200 mg/m², for example 125-175 mg/m²) when given on a weekly schedule. For example, the amount (dose) of a taxane (e.g., paclitaxel) is about 60 to about 300 mg/m² (e.g., about 100 mg/m², 125 mg/m², 150 mg/m², 175 mg/m², 200 mg/m², 225 mg/m², 250 mg/m², or 260 mg/m²) on a three week schedule. In some embodiments, the amount (dose) of a taxane (e.g., paclitaxel) is about 60 to about 300 mg/m² (e.g., about 100 mg/m², 125 mg/m², 150 mg/m², 175 mg/m², 200 mg/m², 225 mg/m², 250 mg/m², or 260 mg/m²) administered weekly. In some embodiments, the amount (dose) of a taxane (e.g., paclitaxel) is about 60 to about 300 mg/m² (e.g., about 100 mg/m², 125 mg/m², 150 mg/m², 175 mg/m², 200 mg/m², 225 mg/m², 250 mg/m², or 260 mg/m²) administered weekly for three out of a four week schedule.

Other exemplary dosing schedules for the administration of the nanoparticle composition (e.g., paclitaxel/albumin nanoparticle composition) include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 150 mg/m², weekly, 3 out of 4 weeks; 175 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week, 50-70 mg/m² twice a week, 50-70 mg/m² three times a week, 30-70 mg/m² daily. The dosing frequency of the composition may be adjusted over the course of the treatment based on the judgment of the administering physician.

In some embodiments, the cancer is breast cancer and the dose of the taxane in the nanoparticle composition is about 180 to about 260 mg/m², such as 260 mg/m², once every weeks.

In some embodiments, the cancer is pancreatic cancer and the dose of the taxane in the nanoparticle composition is about 75 mg/m² to about 125 mg/m², such as 100 mg/m², weekly, three out of four weeks.

In some embodiments, the cancer is melanoma and the dose of the taxane in the nanoparticle composition is about 75 mg/m² to about 125 mg/m², such as 100 mg/m², weekly, three out of four weeks.

In some embodiments, the cancer is lung cancer, and the dose of the taxane in the nanoparticle composition is about 75 mg/m² to about 125 mg/m², such as 100 mg/m², weekly, three out of four weeks.

In some embodiments, the individual is treated for at least about any of one, two, three, four, five, six, seven, eight, nine, or ten treatment cycles.

The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

Other exemplary doses of the taxane (in some embodiments paclitaxel) in the nanoparticle composition include, but are not limited to, about any of 50 mg/m², 60 mg/m², 75 mg/m², 80 mg/m², 90 mg/m², 100 mg/m², 120 mg/m², 140 mg/m², 150 mg/m², 160 mg/m², 175 mg/m², 200 mg/m², 210 mg/m², 220 mg/m², 260 mg/m², and 300 mg/m². For example, the dosage of paclitaxel in a nanoparticle composition can be in the range of about 100-400 mg/m² when given on a 3 week schedule, or about 50-250 mg/m² when given on a weekly schedule.

Therapeutic agent (such as gemcitabine) administered to an individual according to a method described herein may be in the range of about 100 mg/m² to about 5000 mg/m², about 100 mg/m² to about 2000 mg/m², about 200 to about 4000 mg/m², about 300 to about 3000 mg/m², about 400 to about 2000 mg/m², about 500 to about 1500 mg/m², about 500 mg/m² to about 2000 mg/m² about 750 to about 1500 mg/m², about 800 to about 1500 mg/m², about 900 to about 1400 mg/m², about 900 to about 1250 mg/m², about 1000 to about 1500 mg/m², about 800 mg/m², about 850 mg/m², about 900 mg/m², about 950 mg/m², about 1000 mg/m², about 1050 mg/m², about 1100 mg/m², about 1150 mg/m², about 1200 mg/m², about 1250 mg/m², about 1300 mg/m², about 1350 mg/m², about 1400 mg/m², about 1450 mg/m², 1500 mg/m², 1550 mg/m², 1600 mg/m², 1700 mg/m², 1800 mg/m², 1900 mg/m², or 2000 mg/m². Therapeutic agent (such as gemcitabine) may be administered by intravenous (IV) infusion, e.g., over a period of about 10 to about 300 minutes, about 15 to about 180 minutes, about 20 to about 60 minutes, about 10 minutes, about 20 minutes, or about 30 minutes.

Exemplary dosing frequencies for the administration of therapeutic agent (such as gemcitabine) include, but are not limited to, daily, every two days, every three days, every four days, every five days, every six days, weekly without break, weekly for three out of four weeks, once every three weeks, once every two weeks, or two out of three weeks. In some embodiments, therapeutic agent (such as gemcitabine) is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the therapeutic agent (such as gemcitabine) is administered over a period of at least ten days, wherein the interval between each administration is no more than about two days, and wherein the dose of the therapeutic agent (such as gemcitabine) at each administration is about 0.25 mg/m² to about 1500 mg/m², about 10 mg/m² to about 1000 mg/m², about 25 mg/m² to about 750 mg/m², such as about 25 mg/m² to about 500 mg/m², about 25 mg/m² to about 250 mg/m², or about 25 mg/m² to about 100 mg/m².

Other exemplary amounts of therapeutic agent (such as gemcitabine) include, but are not limited to, any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, about 450 to about 500 mg, about 500 to about 600 mg, about 600 to about 700 mg, about 700 to about 800 mg, about 800 to about 900 mg, about 900 to about 1000 mg, about 1000 to about 1250 mg, or about 1250 to about 1500 mg.

The administration of therapeutic agent (such as gemcitabine) can be extended over an extended period of time, such as from about a month up to about seven years. In some embodiments, therapeutic agent (such as gemcitabine) is administered over a period of at least about any of 2, 3, 4, 5, 6, 7, 8, 9, 10, 11, 12, 18, 24, 30, 36, 48, 60, 72, or 84 months.

The composition comprising nanoparticles comprising a taxane (such as paclitaxel) (also referred to as “nanoparticle composition”) and therapeutic agent (such as gemcitabine) can be administered simultaneously (i.e., simultaneous administration) and/or sequentially (i.e., sequential administration).

In some embodiments, the nanoparticle composition and therapeutic agent (such as gemcitabine) are administered simultaneously. The term “simultaneous administration,” as used herein, means that the nanoparticle composition and the other agent are administered with a time separation of no more than about 15 minute(s), such as no more than about any of 10, 5, or 1 minutes. When the drugs are administered simultaneously, the drug in the nanoparticles and the other agent may be contained in the same composition (e.g., a composition comprising both the nanoparticles and the other agent) or in separate compositions (e.g., the nanoparticles are contained in one composition and the other agent is contained in another composition).

In some embodiments, the nanoparticle composition and therapeutic agent (such as gemcitabine) are administered sequentially. The term “sequential administration” as used herein means that the drug in the nanoparticle composition and the other agent are administered with a time separation of more than about 15 minutes, such as more than about any of 20, 30, 40, 50, 60 or more minutes. Either the nanoparticle composition or the other agent may be administered first. The nanoparticle composition and the other agent are contained in separate compositions, which may be contained in the same or different packages.

In some embodiments, the administration of the nanoparticle composition and therapeutic agent (such as gemcitabine) are concurrent, i.e., the administration period of the nanoparticle composition and that of therapeutic agent (such as gemcitabine) overlap with each other. In some embodiments, the nanoparticle composition is administered for at least one cycle (for example, at least any of 2, 3, or 4 cycles) prior to the administration of therapeutic agent (such as gemcitabine). In some embodiments, therapeutic agent (such as gemcitabine) is administered for at least any of one, two, three, or four weeks. In some embodiments, the administrations of the nanoparticle composition and therapeutic agent (such as gemcitabine) are initiated at about the same time (for example, within any one of 1, 2, 3, 4, 5, 6, or 7 days). In some embodiments, the administrations of the nanoparticle composition and therapeutic agent (such as gemcitabine) are terminated at about the same time (for example, within any one of 1, 2, 3, 4, 5, 6, or 7 days). In some embodiments, the administration of therapeutic agent (such as gemcitabine) continues (for example for about any one of 1, 2, 3, 4, 5, 6, 7, 8, 9, 10, 11, or 12 months) after the termination of the administration of the nanoparticle composition. In some embodiments, the administration of therapeutic agent (such as gemcitabine) is initiated after (for example after about any one of 1, 2, 3, 4, 5, 6, 7, 8, 9, 10, 11, or 12 months) the initiation of the administration of the nanoparticle composition. In some embodiments, the administrations of the nanoparticle composition and therapeutic agent (such as gemcitabine) are initiated and terminated at about the same time. In some embodiments, the administrations of the nanoparticle composition and therapeutic agent (such as gemcitabine) are initiated at about the same time and the administration of therapeutic agent (such as gemcitabine) continues (for example for about any one of 1, 2, 3, 4, 5, 6, 7, 8, 9, 10, 11, or 12 months) after the termination of the administration of the nanoparticle composition. In some embodiments, the administration of the nanoparticle composition and therapeutic agent (such as gemcitabine) stop at about the same time and the administration of therapeutic agent (such as gemcitabine) is initiated after (for example after about any one of 1, 2, 3, 4, 5, 6, 7, 8, 9, 10, 11, or 12 months) the initiation of the administration of the nanoparticle composition.

In some embodiments, the method comprises more than one treatment cycle, wherein at least one of the treatment cycles comprises the administration of (a) an effective amount of a composition comprising nanoparticles comprising a taxane (such as paclitaxel) and a carrier protein (e.g., albumin); and (b) an effective amount of therapeutic agent (such as gemcitabine). In some embodiments, the treatment cycle comprises no less than about (such as about) 21 days (e.g., 4 weeks). In some embodiments, the treatment cycle comprises less than about 21 days (for example weekly or daily). In some embodiments, the treatment cycle comprises about 28 days.

In some embodiments, the administration of the nanoparticle composition and therapeutic agent (such as gemcitabine) are non-concurrent. For example, in some embodiments, the administration of the nanoparticle composition is terminated before therapeutic agent (such as gemcitabine) is administered. In some embodiments, the administration of therapeutic agent (such as gemcitabine) is terminated before the nanoparticle composition is administered. The time period between these two non-concurrent administrations can range from about two to eight weeks, such as about four weeks.

The dosing frequency of the drug-containing nanoparticle composition and therapeutic agent (such as gemcitabine) may be adjusted over the course of the treatment, based on the judgment of the administering physician. When administered separately, the drug-containing nanoparticle composition and therapeutic agent (such as gemcitabine) can be administered at different dosing frequency or intervals. For example, the drug-containing nanoparticle composition can be administered weekly, while therapeutic agent (such as gemcitabine) can be administered more or less frequently. In some embodiments, sustained continuous release formulation of the drug-containing nanoparticle and/or therapeutic agent (such as gemcitabine) may be used. Various formulations and devices for achieving sustained release are known in the art. Exemplary dosing frequencies are further provided herein.

The nanoparticle composition and therapeutic agent (such as gemcitabine) can be administered using the same route of administration or different routes of administration. Exemplary administration routes are further provided herein. In some embodiments (for both simultaneous and sequential administrations), the taxane (such as paclitaxel) in the nanoparticle composition and therapeutic agent (such as gemcitabine) are administered at a predetermined ratio. For example, in some embodiments, the ratio by weight of the taxane (such as paclitaxel) in the nanoparticle composition and therapeutic agent (such as gemcitabine) is about 1 to 1. In some embodiments, the weight ratio may be between about 0.001 to about 1 and about 1000 to about 1, or between about 0.01 to about 1 and 100 to about 1. In some embodiments, the ratio by weight of the taxane (such as paclitaxel) in the nanoparticle composition and therapeutic agent (such as gemcitabine) is less than about any of 100:1, 50:1, 30:1, 10:1, 9:1, 8:1, 7:1, 6:1, 5:1, 4:1, 3:1, 2:1, and 1:1 In some embodiments, the ratio by weight of the taxane (such as paclitaxel) in the nanoparticle composition and therapeutic agent (such as gemcitabine) is more than about any of 1:1, 2:1, 3:1, 4:1, 5:1, 6:1, 7:1, 8:1, 9:1, 30:1, 50:1, 100:1. Other ratios are contemplated.

The doses required for the taxane (such as paclitaxel) and/or therapeutic agent (such as gemcitabine) may be lower than what is normally required when each agent is administered alone. Thus, in some embodiments, a subtherapeutic amount of the drug in the nanoparticle composition and/or therapeutic agent (such as gemcitabine) are administered. “Subtherapeutic amount” or “subtherapeutic level” refer to an amount that is less than therapeutic amount, that is, less than the amount normally used when the drug in the nanoparticle composition and/or therapeutic agent (such as gemcitabine) are administered alone. The reduction may be reflected in terms of the amount administered at a given administration and/or the amount administered over a given period of time (reduced frequency).

In some embodiments, enough therapeutic agent (such as gemcitabine) is administered so as to allow reduction of the normal dose of the drug in the nanoparticle composition required to effect the same degree of treatment by at least about any of 5%, 10%, 20%, 30%, 50%, 60%, 70%, 80%, 90%, or more. In some embodiments, enough taxane (such as paclitaxel) in the nanoparticle composition is administered so as to allow reduction of the normal dose of therapeutic agent (such as gemcitabine) required to effect the same degree of treatment by at least about any of 5%, 10%, 20%, 30%, 50%, 60%, 70%, 80%, 90%, or more.

In some embodiments, the dose of both the taxane (such as paclitaxel) in the nanoparticle composition and therapeutic agent (such as gemcitabine) are reduced as compared to the corresponding normal dose of each when administered alone. In some embodiments, both the taxane (such as paclitaxel) in the nanoparticle composition and therapeutic agent (such as gemcitabine) are administered at a subtherapeutic, i.e., reduced, level. In some embodiments, the dose of the nanoparticle composition and/or therapeutic agent (such as gemcitabine) is substantially less than the established maximum toxic dose (MTD). For example, the dose of the nanoparticle composition and/or therapeutic agent (such as gemcitabine) is less than about 50%, 40%, 30%, 20%, or 10% of the MTD.

In some embodiments, the dose of taxane (such as paclitaxel) and/or the dose of therapeutic agent (such as gemcitabine) is higher than what is normally required when each agent is administered alone. For example, in some embodiments, the dose of the nanoparticle composition and/or therapeutic agent (such as gemcitabine) is substantially higher than the established maximum toxic dose (MTD). For example, the dose of the nanoparticle composition and/or therapeutic agent (such as gemcitabine) is more than about 50%, 40%, 30%, 20%, or 10% of the MTD of the agent when administered alone.

As will be understood by those of ordinary skill in the art, the appropriate doses of therapeutic agent (such as gemcitabine) will be approximately those already employed in clinical therapies wherein the therapeutic agent (such as gemcitabine) is administered alone or in combination with other agents. Variation in dosage will likely occur depending on the condition being treated. As described above, in some embodiments, therapeutic agent (such as gemcitabine) may be administered at a reduced level.

The nanoparticle compositions and/or therapeutic agent (such as gemcitabine) can be administered to an individual (such as human) via various routes, including, for example, parenteral, intravenous, intraventricular, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition and/or therapeutic agent (such as gemcitabine) may be used. In some embodiments, the composition (and/or therapeutic agent (such as gemcitabine)) is administered intravenously. In some embodiments, the composition (and/or therapeutic agent (such as gemcitabine)) is administered intraportally. In some embodiments, the composition (and/or therapeutic agent (such as gemcitabine)) is administered intraarterially. In some embodiments, the composition (and/or therapeutic agent (such as gemcitabine)) is administered intraperitoneally. In some embodiments, the composition (and/or therapeutic agent (such as gemcitabine)) is administered intrathecally. In some embodiments, the composition (and/or therapeutic agent (such as gemcitabine)) is administered through a ported catheter to spinal fluid. In some embodiments, the composition (and/or therapeutic agent (such as gemcitabine)) is administered intraventricularly. In some embodiments, the composition (and/or therapeutic agent (such as gemcitabine)) is administered systemically. In some embodiments, the composition (and/or therapeutic agent (such as gemcitabine)) is administered by infusion. In some embodiments, the composition (and/or therapeutic agent (such as gemcitabine)) is administered by infusion through implanted pump. In some embodiments, the composition (and/or therapeutic agent (such as gemcitabine)) is administered by a ventricular catheter. In some embodiments, the composition (and/or therapeutic agent (such as gemcitabine)) is administered through a port or portacath. In some embodiments, the port or portacath is inserted into a vein (such as jugular vein, subclavian vein, or superior vena cava).

In some embodiments, there is provided a method of treating pancreatic cancer (e.g., metastatic pancreatic adenocarcinoma) in an individual comprising administering to the individual (a) an effective amount of a composition comprising nanoparticles comprising a taxane (such as paclitaxel) and a carrier protein; and (b) an effective amount of therapeutic agent (such as gemcitabine), wherein the dose of taxane (such as paclitaxel) in the nanoparticle composition is between about 50 mg/m² to about 400 mg/m² (including for example about 100 mg/m² to about 300 mg/m², about 100 mg/m² to about 200 mg/m², or about 100 mg/m² to about 150 mg/m², or about 100 mg/m², or about 125 mg/m², or about 150 mg/m²) and the amount (dose) of therapeutic agent (such as gemcitabine) is about 500 mg/m² to about 2000 mg/m² (for example, about 750 mg/m² to about 1500 mg/m², about 800 mg/m² to about 1200 mg/m², about 750 mg/m², about 1000 mg/m², about 1250 mg/m², or about 1500 mg/m²). In some embodiments, the nanoparticle composition is administered weekly for three weeks of four weeks or weekly. In some embodiments, therapeutic agent (such as gemcitabine) is administered weekly for three weeks of four weeks or weekly.

In some embodiments, the therapeutic agent is gemcitabine. In some embodiments, the dose of paclitaxel in the nanoparticle composition is about 125 mg/m² on days 1, 8, and 15 of each 28 day cycle, and the dose of gemcitabine is about 1000 mg/m² on days 1, 8, and 15 of each 28 day cycle. In some embodiments, the gemcitabine is administered immediately after the completion of the administration of the nanoparticle composition.

A combination of the administration configurations described herein can be used. A method described herein may be performed alone or in conjunction with an additional therapy, such as chemotherapy, radiation therapy, surgery, hormone therapy, gene therapy, immunotherapy, chemoimmunotherapy, cryotherapy, ultrasound therapy, liver transplantation, local ablative therapy, radiofrequency ablation therapy, photodynamic therapy, and the like.

### Nanoparticle Compositions

The nanoparticle compositions described herein may comprise nanoparticles comprising (in various embodiments consisting essentially of) taxane (e.g., paclitaxel) and an albumin (such as human serum albumin). Nanoparticles of poorly water soluble drugs (such as taxane) have been disclosed in, for example, U.S. Pat. Nos. 5,916,596; 6,506,405; 6,749,868, and 6,537,579 and also in U.S. Pat. Pub. Nos. 2005/0004002, 2006/0263434, and 2007/0082838; PCT Patent Application WO08/137148, each of which is incorporated by reference in their entirety. In some embodiments, the poorly water insoluble drug is a taxane (such as paclitaxel or docetaxel).

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprise taxane (e.g., paclitaxel) coated with an albumin (e.g., human serum albumin). In some embodiments, the composition comprises taxane (e.g., paclitaxel) in both nanoparticle and non-nanoparticle forms, wherein at least about any one of 50%, 60%, 70%, 80%, 90%, 95%, or 99% of taxane (e.g., paclitaxel) in the composition are in nanoparticle form. In some embodiments, taxane (e.g., paclitaxel) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of taxane (e.g., paclitaxel) that is substantially free of polymeric materials (such as polymeric matrix).

In some embodiments, the composition comprises albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of albumin (such as human serum albumin) and taxane (e.g., paclitaxel) in the nanoparticle composition is about 18:1 or less, such as about 15:1 or less, for example about 10:1 or less. In some embodiments, the weight ratio of albumin (such as human serum albumin) and taxane (e.g., paclitaxel) in the composition falls within the range of any one of about 1:1 to about 18:1, about 2:1 to about 15:1, about 3:1 to about 13:1, about 4:1 to about 12:1, or about 5:1 to about 10:1. In some embodiments, the weight ratio of albumin and taxane (e.g., paclitaxel) in the nanoparticle portion of the composition is about any one of 1:2, 1:3, 1:4, 1:5, 1:10, 1:15, or less. In some embodiments, the weight ratio of the albumin (such as human serum albumin) and taxane (e.g., paclitaxel) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, or about 1:1 to about 1:1.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises human serum albumin. In some embodiments, the albumin (e.g., HSA) is recombinant albumin. Human serum albumin (HSA) is a highly soluble globular protein of M. 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977)) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context).

Human serum albumin (HSA) has multiple hydrophobic binding sites (a total of eight for fatty acids, an endogenous ligand of HSA) and binds a diverse set of taxanes, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Paclitaxel has been shown to bind HSA (see, e.g., Paal et al., Eur. J. Biochem., 268(7), 2187-91 (200a)).

The albumin (such as human serum albumin) in the composition generally serves as a carrier for taxane (e.g., paclitaxel), i.e., the albumin in the composition makes taxane (e.g., paclitaxel) more readily suspendable in an aqueous medium or helps maintain the suspension as compared to compositions not comprising an albumin. This can avoid the use of toxic solvents (or surfactants) for solubilizing taxane (e.g., paclitaxel), and thereby can reduce one or more side effects of administration of taxane (e.g., paclitaxel) into an individual (such as a human). Thus, in some embodiments, the composition described herein is substantially free (such as free) of surfactants, such as Cremophor (including Cremophor EL® (BASF)). In some embodiments, the nanoparticle composition is substantially free (such as free) of surfactants. A composition is “substantially free of Cremophor” or “substantially free of surfactant” if the amount of Cremophor or surfactant in the composition is not sufficient to cause one or more side effect(s) in an individual when the nanoparticle composition is administered to the individual. In some embodiments, the nanoparticle composition contains less than about any one of 20%, 15%, 10%, 7.5%, 5%, 2.5%, or 1% organic solvent or surfactant.

The amount of albumin in the composition described herein will vary depending on other components in the composition. In some embodiments, the composition comprises an albumin in an amount that is sufficient to stabilize taxane (e.g., paclitaxel) in an aqueous suspension, for example, in the form of a stable colloidal suspension (such as a stable suspension of nanoparticles). In some embodiments, the albumin is in an amount that reduces the sedimentation rate of taxane (e.g., paclitaxel) in an aqueous medium. For particle-containing compositions, the amount of the albumin also depends on the size and density of nanoparticles of taxane (e.g., paclitaxel).

Taxane (e.g., paclitaxel) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize taxane (e.g., paclitaxel) in an aqueous suspension at a certain concentration. For example, the concentration of taxane (e.g., paclitaxel) in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of taxane (e.g., paclitaxel) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of albumin. In some embodiments, the composition, in liquid form, comprises about 0.5% to about 5% (w/v) of albumin.

In some embodiments, the weight ratio of albumin, e.g., albumin, to taxane (e.g., paclitaxel) in the nanoparticle composition is such that a sufficient amount of taxane (e.g., paclitaxel) binds to, or is transported by, the cell. While the weight ratio of albumin to taxane (e.g., paclitaxel) will have to be optimized for different albumin and taxane (e.g., paclitaxel) combinations, generally the weight ratio of albumin, e.g., albumin, to taxane (e.g., paclitaxel) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to taxane (e.g., paclitaxel) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human serum albumin) and taxane (e.g., paclitaxel) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, or about 1:1 to about 1:1.

In some embodiments, the albumin allows the composition to be administered to an individual (such as human) without significant side effects. In some embodiments, the albumin (such as human serum albumin) is in an amount that is effective to reduce one or more side effects of administration of taxane (e.g., paclitaxel) to a human. The term “reducing one or more side effects of administration” refers to reduction, alleviation, elimination, or avoidance of one or more undesirable effects caused by taxane (e.g., paclitaxel), as well as side effects caused by delivery vehicles (such as solvents that render taxane (e.g., paclitaxel) suitable for injection) used to deliver taxane (e.g., paclitaxel). In some embodiments, the one or more side effects are adverse side effects (AEs). In some embodiments, the one or more side effects are serious adverse side effects (SAEs). Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with taxane (e.g., paclitaxel) can be reduced.

In some embodiments, the nanoparticle compositions described herein comprises nanoparticles comprising a taxane (such as paclitaxel) and an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) and an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) and an albumin (such as human albumin or human serum albumin), wherein the nanoparticles have an average diameter of about 130 nm. In some embodiments, the nanoparticle compositions described herein comprises nanoparticles comprising paclitaxel and human albumin (such as human serum albumin), wherein the nanoparticles have an average diameter of about 130 nm.

In some embodiments, the nanoparticle compositions described herein comprises nanoparticles comprising a taxane (such as paclitaxel) and an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) and an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) and an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel and human albumin (such as human serum albumin), wherein the nanoparticles have an average diameter of about 130 nm, wherein the weight ratio of albumin and the taxane in the composition is about 9:1.

In some embodiments, the nanoparticle compositions described herein comprises nanoparticles comprising a taxane (such as paclitaxel) coated with an albumin (such as human albumin or human serum albumin). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) coated with an albumin (such as human albumin or human serum albumin), wherein the nanoparticles have an average diameter of about 130 nm. In some embodiments, the nanoparticle compositions described herein comprises nanoparticles comprising paclitaxel coated with human albumin (such as human serum albumin), wherein the nanoparticles have an average diameter of about 130 nm.

In some embodiments, the nanoparticle compositions described herein comprises nanoparticles comprising a taxane (such as paclitaxel) coated with an albumin (such as human albumin or human serum albumin), wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel coated with human albumin (such as human serum albumin), wherein the nanoparticles have an average diameter of about 130 nm, wherein the weight ratio of albumin and the taxane in the composition is about 9:1.

In some embodiments, the nanoparticle compositions described herein comprises nanoparticles comprising a taxane (such as paclitaxel) stabilized by an albumin (such as human albumin or human serum albumin). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) stabilized by an albumin (such as human albumin or human serum albumin), wherein the nanoparticles have an average diameter of about 130 nm. In some embodiments, the nanoparticle compositions described herein comprises nanoparticles comprising paclitaxel stabilized by human albumin (such as human serum albumin), wherein the nanoparticles have an average diameter of about 130 nm.

In some embodiments, the nanoparticle compositions described herein comprises nanoparticles comprising a taxane (such as paclitaxel) stabilized by an albumin (such as human albumin or human serum albumin), wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel stabilized by human albumin (such as human serum albumin), wherein the nanoparticles have an average diameter of about 130 nm, wherein the weight ratio of albumin and the taxane in the composition is about 9:1.

In some embodiments, the nanoparticle composition comprises Abraxane® (Nab-paclitaxel). In some embodiments, the nanoparticle composition is Abraxane® (Nab-paclitaxel). Abraxane® is a formulation of paclitaxel stabilized by human albumin USP, which can be dispersed in directly injectable physiological solution. When dispersed in a suitable aqueous medium such as 0.9% sodium chloride injection or 5% dextrose injection, Abraxane® forms a stable colloidal suspension of paclitaxel. The mean particle size of the nanoparticles in the colloidal suspension is about 130 nanometers. Since HSA is freely soluble in water, Abraxane® can be reconstituted in a wide range of concentrations ranging from dilute (0.1 mg/ml paclitaxel) to concentrated (20 mg/ml paclitaxel), including for example about 2 mg/ml to about 8 mg/ml, about 5 mg/ml.

Methods of making nanoparticle compositions are known in the art. For example, nanoparticles containing taxane (e.g., paclitaxel) and albumin (such as human serum albumin) can be prepared under conditions of high shear forces (e.g., sonication, high pressure homogenization, or the like). These methods are disclosed in, for example, U.S. Pat. Nos. 5,916,596; 6,506,405; 6,749,868, and 6,537,579 and also in U.S. Pat. Pub. No. 2005/0004002, 2007/0082838, 2006/0263434 and PCT Application WO08/137148 and WO08/109163.

Briefly, taxane (e.g., paclitaxel) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (e.g., with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1.

### Other Components in the Nanoparticle Compositions

The nanoparticles described herein can be present in a composition that includes other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the nanoparticle composition (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Kits, Medicines, and Compositions

The invention also provides kits, medicines, compositions, and unit dosage forms for use in any of the methods described herein.

Kits of the invention include one or more containers comprising taxane (e.g., paclitaxel)-containing nanoparticle compositions (or unit dosage forms and/or articles of manufacture) and/or a therapeutic agent (e.g., gemcitabine), and in some embodiments, further comprise instructions for use in accordance with any of the methods described herein including methods for treating, assessing responsiveness, monitoring, identifying individuals, and selecting patients for treatment comprising a) nanoparticles comprising a taxane and an albumin and/or b) a therapeutic agent (e.g., gemcitabine) based upon a K-ras mutation status determined at one or more K-ras positions selected from the group consisting of G12, G13, S17, P34, Q61, K117, or A146 of the K-ras amino acid sequence of SEQ ID NO:2 in a sample.

Kits of the also invention include one or more containers comprising taxane (e.g., paclitaxel)-containing nanoparticle compositions (or unit dosage forms and/or articles of manufacture) and/or a therapeutic agent (e.g., gemcitabine), and in some embodiments, further comprise instructions for use in accordance with any of the methods described herein including methods for treating, assessing responsiveness, monitoring, identifying individuals, and selecting patients for treatment comprising a) nanoparticles comprising a taxane and an albumin and/or b) a therapeutic agent (e.g., gemcitabine) based upon a K-ras mutation status determined at one or more K-ras positions selected from the group consisting of G12, G13, S17, P34, or Q61 of the K-ras amino acid sequence of SEQ ID NO:2 in a sample.

Kits of the also invention include one or more containers comprising taxane (e.g., paclitaxel)-containing nanoparticle compositions (or unit dosage forms and/or articles of manufacture) and/or a therapeutic agent (e.g., gemcitabine), and in some embodiments, further comprise instructions for use in accordance with any of the methods described herein including methods for treating, assessing responsiveness, monitoring, identifying individuals, and selecting patients for treatment comprising a) nanoparticles comprising a taxane and an albumin and/or b) a therapeutic agent (e.g., gemcitabine) based upon a K-ras mutation status determined at one or more K-ras positions selected from the group consisting of G12, G13, Q61, K117 or A146 of the K-ras amino acid sequence of SEQ ID NO:2 in a sample.

Kits of the also invention include one or more containers comprising taxane (e.g., paclitaxel)-containing nanoparticle compositions (or unit dosage forms and/or articles of manufacture) and/or a therapeutic agent (e.g., gemcitabine), and in some embodiments, further comprise instructions for use in accordance with any of the methods described herein including methods for treating, assessing responsiveness, monitoring, identifying individuals, and selecting patients for treatment comprising a) nanoparticles comprising a taxane and an albumin and/or b) a therapeutic agent (e.g., gemcitabine) based upon a K-ras mutation status determined at one or more K-ras positions selected from the group consisting of G12, G13, or Q61 of the K-ras amino acid sequence of SEQ ID NO:2 in a sample.

Kits of the invention also include one or more containers comprising taxane (e.g., paclitaxel)-containing nanoparticle compositions (or unit dosage forms and/or articles of manufacture) and/or a therapeutic agent (e.g., gemcitabine), and in some embodiments, further comprise instructions for use in accordance with any of the methods described herein including methods for treating, assessing responsiveness, monitoring, identifying individuals, and selecting patients for treatment comprising a) nanoparticles comprising a taxane and an albumin and/or b) a therapeutic agent (e.g., gemcitabine), based upon a K-ras mutation status determined at one or more K-ras positions selected from Table 1. The kit may comprise a description of selection of an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a) a composition comprising nanoparticles comprising taxane (e.g., paclitaxel) and an albumin (such as human serum albumin), b) an effective amount of a therapeutic agent (e.g., gemcitabine), and c) instructions for screening a K-ras mutation selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In other embodiments, the K-ras mutation selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In yet other embodiments, the K-ras mutation selected from the group consisting of G12C, G12R, G12S, G12A, G12D, G12V, G13C, G13R, G13S, G13A, G13D, Q61K, Q61L, Q61R and Q61H.

The nanoparticles and the therapeutic agent (e.g., gemcitabine) can be present in separate containers or in a single container. For example, the kit may comprise one distinct composition or two or more compositions wherein one composition comprises nanoparticles and one composition comprises therapeutic agent (e.g., gemcitabine).

The kits of the invention are in suitable packaging. Suitable packaging include, but is not limited to, vials, bottles, jars, flexible packaging (e.g., Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions may also comprise instructions relating to the use of the taxane (e.g., paclitaxel) nanoparticle compositions and the therapeutic agent (e.g., gemcitabine) generally include information as to dosage, dosing schedule, and route of administration for the intended treatment. In some embodiments, the dosage of taxane (e.g., paclitaxel) in nanoparticle composition is between about 50 to about 125 mg/m² and the dosage of therapeutic agent (e.g., gemcitabine) is between about 5 mg/kg to about 60 mg/kg. In some embodiments, the amount (dose) of gemcitabine is between about 500 mg/m² to about 2000 mg/m², including for example about 75 mg/m² to about 1500 mg/m², about 800 mg/m² to about 1200 mg/m², about 750 mg/m², about 1000 mg/m², about 1250 mg/m², about 1500 mg/m², or about 2000 mg/m². In some embodiments, the dosage of taxane (e.g., paclitaxel) in nanoparticle composition is between about 50 to about 125 mg/m² weekly and the dosage of therapeutic agent (e.g., gemcitabine) is between about 500 mg/m² to about 2000 mg/m² (for example about 75 mg/m² to about 1500 mg/m² or about 1000 mg/m²), once every week. In some embodiments, the dosage of taxane (e.g., paclitaxel) in nanoparticle composition is about 125 mg/m² weekly, three out of four weeks and the dosage of therapeutic agent (e.g., gemcitabine) is about 1000 mg/m² weekly, three out of 4 weeks.

In some embodiments, the taxane (e.g., paclitaxel) nanoparticle composition and/or the therapeutic agent (e.g., gemcitabine) is administered intravenously. In some embodiments, the taxane (e.g., paclitaxel) nanoparticle composition and therapeutic agent (e.g., gemcitabine) are administered intravenously. In some embodiments, the therapeutic agent is gemcitabine. In some embodiments, the instructions indicate that taxane (e.g., paclitaxel) nanoparticle composition and/or the therapeutic agent (e.g., gemcitabine) is administered intravenously. In some embodiments, the instructions indicate that taxane (e.g., paclitaxel) nanoparticle composition and the therapeutic agent (e.g., gemcitabine) are administered intravenously. In some embodiments, the instructions indicate that the therapeutic agent is gemcitabine.

The containers may be unit doses, bulk packages (e.g., multi-dose packages) or sub-unit doses. For example, kits may be provided that contain sufficient dosages of taxane (e.g., paclitaxel) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more.

Kits may also include multiple unit doses of taxane (e.g., paclitaxel) and pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein. In some embodiments, there is provided a medicine (or composition or a unit dosage form) for use in treating cancer in conjunction with the therapeutic agent (e.g., gemcitabine), comprising nanoparticles comprising taxane (e.g., paclitaxel) and an albumin (such as human serum albumin), wherein the therapeutic agent (e.g., gemcitabine). In some embodiments, there is provided a medicine (or composition or a unit dosage form) for use in treating cancer, comprising nanoparticles comprising taxane (e.g., paclitaxel) and an albumin (such as human serum albumin) and the therapeutic agent (e.g., gemcitabine).

### EXAMPLES

### Example 1

### Method of Treating Metastatic Pancreatic Cancer in Patients Having a K-ras Mutation

Patients diagnosed with metastatic pancreatic cancer are assessed for K-ras mutation status. Specifically, a biopsy sample from the patient is processed for sequence analysis to determine the mutation status at one or more of the following amino acid locations on K-ras: G12, G13 S17, P34 or Q61. The patient is identified for treatment if the patient has one or more of the following mutations: G12C, G12S, G12R, G12F, G12L, G12N, G12A, G12D, G12V, G13C, G13S, G13D, G13V, G13P, S17G, P34S, Q61K, Q61L, Q61R, and Q61H. Abraxane and gemcitabine are then administered to the patient at the dose of 125 mg/m² on days 1, 8, 15 of a four week cycle.

### Example 2

### Methods of Treating Melanoma in Patients Having K-ras Mutation

Patients diagnosed with melanoma are assessed for K-ras mutation status. Specifically, a biopsy sample from the patient is processed for sequence analysis to determine the mutation status at one or more of the following amino acid locations on K-ras: G12, G13, Q61, or F156. The patient is identified for treatment if the patient has one or more of the following mutations: G12C, G12R, G12S, G12D, G12V, G13D, Q61K, Q61R, Q61L, Q61H, and F156L. Abraxane and gemcitabine are then administered to the patient at the dose of 100 mg/m² on days 1, 8, 15 of a four week cycle.

### Example 3

### Method of Treating Non-Small Cell Lung Cancer in Patients Having K-ras Mutation

Patients diagnosed with non-small cell lung cancer are assessed for K-ras mutation status. Specifically, a biopsy sample from the patient is processed for sequence analysis to determine the mutation status at one or more of the following amino acid locations on K-ras: G12, G13 or Q61. The patient is identified for treatment if the patient has one or more of the following mutations: G12C, G12R, G12S, G12A, G12D, G12V, G13C, G13R, G13S, G13A, G13D, Q61K, Q61L, Q61R and Q61H. Abraxane and gemcitabine are then administered to the patient at the dose of 100 mg/m² on days 1, 8, 15 of a four week cycle.

### Example 4

### Method of Treating Colorectal Cancer in Patients Having K-ras Mutation

Patients diagnosed with colorectal cancer are assessed for K-ras mutation status. Specifically, a biopsy sample from the patient is processed for sequence analysis to determine the mutation status at one or more of the following amino acid locations on K-ras: G12, G13, Q61, K117 or A146. The patient is identified for treatment if the patient has one or more of the following mutations: G12C, G12R, G12S, G12A, G12D, G12V, G13C, G13R, G13S, G13A, G13D, G13V, Q61K, Q61L, Q61R, Q61H, K117N, A146P, A146T and A146V. Abraxane and gemcitabine are then administered to the patient at the dose of 100 mg/m² on days 1, 8, 15 of a four week cycle.

### Example 5

### Method of Treating Breast Cancer in Patients Having K-ras Mutation

Patients diagnosed with breast cancer are assessed for K-ras mutation status. Specifically, a biopsy sample from the patient is processed for sequence analysis to determine the mutation status at one or more of the following amino acid locations on K-ras: G12, G13, or Q61. The patient is identified for treatment if the patient has one or more of the following mutations: G12C, G12R, G12A, G12D, G12V, G13D, and Q61L. Abraxane and gemcitabine are then administered to the patient at the dose of 100 mg/m² on days 1, 8, 15 of a four week cycle.

### Example 6

### Method of Ovarian Cancer in Patients Having K-ras Mutation

Patients diagnosed with ovarian are assessed for K-ras mutation status. Specifically, a biopsy sample from the patient is processed for sequence analysis to determine the mutation status at one or more of the following amino acid locations on K-ras: G12 or G13. The patient is identified for treatment if the patient has one or more of the following mutations: G12C, G12R, G12A, G12D, G12V, and G13D. Abraxane and gemcitabine are then administered to the patient at the dose of 100 mg/m² on days 1, 8, 15 of a four week cycle.

What is claimed is:

1. A method of treating cancer in an individual comprising administering to the individual an effective amount of a composition comprising nanoparticles comprising a taxane and an albumin, wherein the K-ras mutation status is used as a basis for selecting the individual for treatment.

2. The method of claim 1, wherein the individual is selected for treatment if the individual has a K-ras mutation.

3. The method of claim 1, wherein the K-ras mutation is at one or more of G12, G13, S17, P34 or Q61 of the K-ras amino acid sequence.

4. The method of claim 3, wherein the K-ras mutation is one or more selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H.

5. The method of claim 1, wherein the K-ras mutation is at one or more of G12, G13, Q61, K117 or A146 of the K-ras amino acid sequence.

6. The method of claim 5, wherein the K-ras mutation is one or more selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V.

7. The method of claim 1, wherein the K-ras mutation is at one or more of G12, G13 or Q61 of the K-ras amino acid sequence.

8. The method of claim 7, wherein the K-ras mutation is one or more selected from the group consisting of G12C, G12R, G12S, G12A, G12D, G12V, G13C, G13R, G13S, G13A, G13D, Q61K, Q61L, Q61R and Q61H.

9. The method of claim 1, wherein the method further comprises administering to the individual an effective amount of a therapeutic agent.

10. The method of claim 9, wherein the therapeutic agent is a chemotherapeutic agent or an antibody.

11. The method of claim 10, wherein the chemotherapeutic agent is gemcitabine.

12. The method of claim 1, wherein the method comprises determining the K-ras mutation status of the individual prior to administering to the individual an effective amount of the composition comprising nanoparticles comprising a taxane and an albumin.

13. The method of claim 1, wherein the composition comprising nanoparticles comprising a taxane and an albumin is administered intravenously.

14. The method of claim 1, wherein the taxane is paclitaxel.

15. The method of claim 1, wherein the nanoparticles in the composition comprises the taxane coated with the albumin.

16. The method of claim 1, wherein the nanoparticles in the composition have an average diameter of less than about 200 nm.

17. The method of claim 1, wherein the albumin is human serum albumin.

18. The method of claim 1, wherein the individual is human.

19. A kit comprising 1) a composition comprising nanoparticles comprising a taxane and an albumin, and 2) an agent for determining the K-ras mutation status.


SEQUENCE LISTING (2 sequences)

SEQ ID NO: 1 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 2 - protein, 188 aa, Homo sapiens [sequence omitted]